SEQUENCE LISTING (93 sequences)

SEQ ID NO: 1 - protein, 37 aa, synthetic construct
  VARIANT 17: note = S or W
  VARIANT 18: note = R or K
  VARIANT 19: note = D or N
  VARIANT 20: note = A, T or K
  VARIANT 21: note = I or V
  VARIANT 23: note = N or D
  VARIANT 24: note = E or Q
  VARIANT 32: note = H or N
  VARIANT 36: note = K or H
PEPGRCRRTDGKKWRCXXXXXPXXKYCERHMXRGRXR

SEQ ID NO: 2 - protein, 31 aa, synthetic construct
  VARIANT 1: note = A, G or C
  VARIANT 2: note = A, P or S
  VARIANT 8: note = L or W
  VARIANT 12: note = E or Q
  VARIANT 13: note = Q or L
  VARIANT 15: note = S or A
  VARIANT 16: note = L or R
  VARIANT 17: note = I or V
  VARIANT 19: note = K or Q
  VARIANT 21: note = M or I
  VARIANT 22: note = A or T
  VARIANT 24: note = R, N or G
XXFTAMQXQELXXQXXXYXYXXAXVPVPTHL

SEQ ID NO: 3 - protein, 38 aa, synthetic construct
  VARIANT 10: note = V or M
  VARIANT 12: note = S or A
  VARIANT 13: note = R or K
  VARIANT 20: note = V or I
  VARIANT 30: note = F or L
  VARIANT 33: note = L or I
GGKDRHSKVXTXXGLRDRRXRLSVPTAIQXYDXQDRLG

SEQ ID NO: 4 - protein, 28 aa, synthetic construct
  VARIANT 3: note = Q or P
  VARIANT 4: note = S or V
  VARIANT 5: note = Q or A
  VARIANT 6: note = N or S
  VARIANT 7: note = P or L
  VARIANT 8: note = S or A
  VARIANT 9: note = A or L
  VARIANT 10: note = A, E or S
  VARIANT 11: note = K or D
  VARIANT 13: note = A or G
  VARIANT 14: note = C or E
  VARIANT 15: note = S or V
  VARIANT 25: note = V or G
ANXXXXXXXXXSXXXSTSETSKGSXLSL

SEQ ID NO: 5 - protein, 50 aa, synthetic construct
  VARIANT 12: note = R or K
  VARIANT 22: note = S or A
RKDRHSKVCTAXGPRDRRVRLXAHTAIQFYDVQDRLGYDRPSKAVDWLIK

SEQ ID NO: 6 - protein, 48 aa, synthetic construct
  VARIANT 2: note = A or P
  VARIANT 7: note = V, A or T
  VARIANT 9: note = A or T
  VARIANT 10: note = H or Q
  VARIANT 18: note = G or S
  VARIANT 29: note = S or N
  VARIANT 30: note = Q or H
  VARIANT 40: note = S or P
EXSSSTXAXXSSAMGFQXYTPDLLSRTGXXSQELRLSLQXLPDPMFHH

SEQ ID NO: 7 - protein, 273 aa, Zea mays
MAAEGEAKNPSGGGEGGNPQHQQAVQAAPAEPPTAQGEAVQEAGAQATGQEPEGEKANRDGEGSAGEKDDGACRDLVLVEDPEVLAVEDPEEAAATAALQEEMKALVASVPDGAGAAFTAMQLQELEQQSRVYQYMAARVPVPTHLVFPVWKSVTGASSEGAQKYPTLLGLATLCLDFGKNPEPEPGRCRRTDGKKWRCWRNTIPNEKYCERHMHRGRKRPVQVVEEAEPDSASGSKSAPSKATEGAKKVDDKSPGSKKLAVAAAAAAAVQST

SEQ ID NO: 8 - protein, 269 aa, Zea mays
MTAEGEAKNPSAGGGGDNPQHQQAAPAPAPAQGEVAQEAAVQGTGQEQERDKADREVQGGAGEKDDGACRDLVLVEDPEVLAVEDPEEAAATAALQEEMKALVASIPDGAGAAFTAMQLQELEQQSRVYQYMAARVPVPTHLVFPVWKSVTGASSEGAQKYPTLMGLATLCLDFGKNPEPEPGRCRRTDGKKWRCWRNTIPNEKYCERHMHRGRKRPVQVFLEDDEPDSASGSKPAAPGKATEGAKKADDKSPSSKKLAVAAPAAVQST

SEQ ID NO: 9 - protein, 530 aa, Arabidopsis thaliana
MDLGVRVSGHETVSSPGQTELGSGFSNKQERSGFDGEDCWRSSKLSRTSTDGFSSSPASAKTLSFHQGIPLLRSTTINDPRKGQEHMLSFSSASGKSDVSPYLQYCRNSGYGLGGMMNTSNMHGNLLTGVKGPFSLTQWAELEQQALIYKYITANVPVPSSLLLSLKKSFFPYGSLPPNSFGWGSFHLGFSGGNMDPEPGRCRRTDGKKWRCSRDAVPDQKYCERHINRGRHRSRKPVEGQNGHNTNAAAAASAAAASTAAAVSKAAAGTSAVAMRGSDNNNSLAAAVGTQHHTNNQSTDSLANRVQNSRGASVFPATMNLQSKETHPKQSNNPFEFGLISSDSLLNPSHKQASYATSSKGFGSYLDFGNQAKHAGNHNNVDSWPEELKSDWTQLSMSIPMAPSSPVQDKLALSPLRLSREFDPAIHMGLGVNTEFLDPGKKTNNWIPISWGNNNSMGGPLGEVLNSTTNSPKFGSSPTGVLQKSTFGSLSNSSSASSTIIGDNNNKNGDGKDPLGPTTLMNTSATAPSL

SEQ ID NO: 10 - protein, 535 aa, Arabidopsis thaliana
MDIGVHVLGSVTSNENESLGLKELIGTKQDRSGFIGEDCLQRSLKLARTTTRAEEEENLSSSVAAAYCKTMSFHQGIPLMRSASPLSSDSRRQEQMLSFSDKPDALDFSKYVGLDNSSNNKNSLSPFLHQIPPPSYFRSSGGYGSGGMMMNMSMQGNFTGVKGPFTLTQWAELEQQALIYKYITANVPVPSSLLISIKKSFYPYGSLPPSSFGWGTFHLGFAGGNMDPEPGRCRRTDGKKWRCSRDAVPDQKYCERHINRGRHRSRKPVEVQSGQNQTAAAASKAVTTPQQPVVAGNTNRSNARASSNRSLAIGSQYINPSTESLPNNRGVSIYPSTVNLQPKESPVIHQKHRNNNNPFEFGHISSDSLLNPNTAKTYGSSFLDFSSNQEKHSGNHNHNSWPEELTSDWTQLSMSIPIASSSPSSTHNNNNAQEKTTLSPLRLSRELDLSIQTDETTIEPTVKKVNTWIPISWGNSLGGPLGEVLNSTTNSPTFGSSPTGVLQKSTFCSLSNNSSVSSPIAENNRHNGDYFHYTT

SEQ ID NO: 11 - protein, 455 aa, Glycine max
MHGALAGVRGPFTPSQWMELEHQALIYKYITSNVPVPTHLIPIRKALDSLGFCNFSTGLLRPNALGWGGFHLGFSNNTDPEPGRCRRTDGKKWRCSRDAVVDQKYCERHMNRGRHRSRKPVEGQLGHALTTTTTTNTPNASSNSTVVPRNTTTTTTFAHNNVHHPLPPHSSQANTINRMFTSNKENNSTSERLQDPALPMLPPTLELKPKENNPFMIHKHHIPADEYSRNSNEFGLVTSDSLLNPTQKRSFTSSQKDDSESQQQHSLRHFIDDSPKPQSHNHHHHHHSSSIWPELDSMQSDRTQLSISIPISSSDFMSFTTSSPSNEKLTLSPLGHSRELDPIQMELGMGSGASNEANTRQANWIPITWESSMGGPLGEVLNLSNNSNASDQCGKNNNTSALNLMKDGWDNNPPLGSSPTGVLQKSAFGSLSNSSAGSSPRAENNKEGATLCNAL

SEQ ID NO: 12 - protein, 600 aa, Glycine max
MDLGVVGLEGVVGSESGCVFGSSLVSDPETKHKWYGSGLLKQERSAIATEDDEWRISKVAKTDHDMSSASKAMLFQQRNNSLLRSNNATLFSDGHHQSQMLSFSSPKSDSLLIDKASSNATLPFSSHQLSSYTRNTGYNSGSISMHGALASVRGPFTPSQWMELEHQALIYKYITANVPVPTHLLIPIRKALDSVGFCNFSAGLLRPNSLGWGGFHLGFSNNTDPEPGRCRRTDGKKWRCSRDAVVDQKYCERHMNRGRHRSRKPVEGQSGHALTTTTSNTPNASSNSVVPGNNNNTFAHNNVHHPIPPHSSPVNTITRMFTSNKENNNSTSERMQDPALPMLPPTLELKPKENNPFMIHKHQIPSDEYSSRNNNEFGLVTSDSLLNPSEKRSFTSSQKNDSSESQQQHSLRHFIDDSPKPQSNHHHRSSSIWPELDNMQSDRTQLSISIPISSSDHFMSFTTSLPSNEKLTLSPLRLSRELDPIQMGLGVGSAPNEANTRQANWIPITWESSMGGPLGEVLNLSNNNNSNASDQCGKNNNNTSALNLMKDGWDNNPPSGSSPTGVLQKSAFGSLSNSSAGSSPRGAENNKEGATLCNAL

SEQ ID NO: 13 - protein, 255 aa, Gossypium raimondii
MNCAHTQEMEPQDSSPLKTANFSDLGIGLTNLGDSWNKESNRLSGGEGSPPIGLGLELGCGSGHIPTGITKSCGFTVFQLQELQLQSLIYKYMEAGLPVPHHLLLPIWKSVAGSLGGLHGSPYQLYYGFLGCGPLQLGYKNGVDPEPGRCRRTDGKKWRCSKEAVPDHKYCERHMHRGRQRSTKLVEASQATRTSISRNNANTNLSISLQVDSSNNSGNGSNLSSSFTGFSPNIALLRGGDSKAVPHSLQFQELL

SEQ ID NO: 14 - protein, 444 aa, Gossypium raimondii
MDAQPVQTRILPDSGGGGSGAQERLEGKGRTVKMEEEETEKGGSPSIKLGLGIDGTTSEKPVKKHGKCVFTTAQLHDLQLQTLIFKYIAGGIQVPVNLVIPIWKSVASSFGSARGCIYERYPSFVGVSPQALDYRNMADPEPGRCRRTDGKKWRCSKNVIPYQKYCEQHMHRGCRRSRKPVETSQSALPDSTSSKFSIRLSENSKNLPTPVSFQYTNPFSCNTSTSHGTTAIESNNVCSNRNSISINAATSGTIIATMKNDNKNDPKRNKNVTNTSEKGEEKLSVSNNNTIKRSSKSGNKATVGNNISPSVGFSPKSVLQVLGNSSSRAYKNEIELEPGRCRRTDGKRWRCSRDVIPDQKYCARHMHRGARKQTEVSQPVAVPSVGECRPPSRLTTAYKAACPALSTSLSISIPRSQYIAEDEKSTSSSSETTISDTNDYCLRE

SEQ ID NO: 15 - protein, 267 aa, Gossypium raimondii
MEPQYTPLKIARFADFGTLFFLYSLSLDYHALVNLLFSFFAGIGLTNMESNMLSSGEGSPPIGLGLELGRGSDHRPTGFTKSCGFTVFQLQELQLQSLIFKYMEAGLPVPNHLLLPIWKSVAGSLGGLHANPYQLYSSYLGCGPLHLEYKNGVDPEPGRCRRTDGKKWRCSKEAVPDHKYCERHMHRGRQRSRKLVEASQATSSGSRIISNGNADTNLSISLQLDNSNTSSSGSSSNLSSGFIGFSPKSVLRGSNPKSESSIQFQEI

SEQ ID NO: 16 - protein, 269 aa, Oryza sativa
MAAEGEAKKDSASNPPGGGGGGGGGEEEEDSSLAVGEAAVGVGEAGGGGGGGEKADREEEEGKEDVEEGGVCKDLVLVEDAVPVEDPEEAAATAALQEEMKALVESVPVGAGAAFTAMQLQELEQQSRVYQYMAARVPVPTHLVFPIWKSVTGASSEGAQKYPTLMGLATLCLDFGKNPEPEPGRCRRTDGKKWRCWRNAIANEKYCERHMHRGRKRPVQLVVEDDEPDSTSGSKPASGKATEGGKKTDDKSSSSKKLAVAAPAAVEST

SEQ ID NO: 17 - protein, 271 aa, Sorghum bicolor
MAAEGEGKNPAGGGGGDNPQHQQAVQAAPAPAAQGEAAQEAGGQGTGLEPEGEKADREGEGGGAGEKDDAACRDLVLVEDPEVVAVEDPEEAAATAALQEEMKALVASIPDGAGAAFTAMQLQELEQQSRVYQYMAARVPVPTHLVFPVWKSVTGASSEGAQKYPTLMGLATLCLDFGKNPEPEPGRCRRTDGKKWRCWRSTIPNEKYCERHMHRGRKRPVQVVVEDDEPDSASGSKSTPGKATDGAKKADDKSPSSKKLAVAAPAAVQST

SEQ ID NO: 18 - protein, 260 aa, Triticum aestivum
MAAEGEDKKDANSVGGGGGGGENTLEAAEEAILQAVGQEPGQELEGEVEESADREGNGDDAGKEDSGCKDLVLVEDPVLVEDPEEALQEEMRALFASVPEGAGASFTAMQLQELEQQSRVYQYMAARVPVPTHLVFPIWKSVTGASSEGAQNYPTLMGLATLCLDFGKSPEPEPGRCRRTDGKKWRCWRKTIPNEKYCERHMHRGRKRPVQVIVEDDEPDSASGSKSSSGKVTEGGKKTDDKSSSSKKLAVAAPAAVEFT

SEQ ID NO: 19 - protein, 263 aa, Triticum aestivum
MAAEGEDKKDANSVGGGGGENTLESAEEAILQAVGQEPGQELEGEAEESADREGNGDDAGKEDSGCKDLVLVEDPVLVEDPEEAVATAAFQEEMRALLASVPEGAGASFTAMQLQELEQQSRVYQYMAARVPVPTHLVFPIWKSVTGASSEGAQNYPTLMGLATLCLDFGKSPEPEPGRCRRTDGKKWRCWRKTIPNEKYCERHMHRGRKRPVQVIVEDDEPDSASGSKSSSGKVTEGGKKTDDKSSSSKKLAVAAPAAVEFT

SEQ ID NO: 20 - protein, 259 aa, Triticum aestivum
MAAEGEDKKDANSVGGGGGGENTLEAAEEAILQAVGQEPGQELEGEAEESADREGNGDDTGKEDSGCKDLVLVEDPVLVEDPEEALQEEMRALLASVPEGAGASFTAMQLQELEQQSRVYQYMAARVPVPTHLVFPIWKSVTGASSEGAQNYPTLMGLATLCLDFGKSPEPEPGRCRRTDGKKWRCWRKTIPNEKYCERHMHRGRKRPVQVIVEDDEPDSASGSKSSSGKVTEGGKKTENKSSSSKKLAVAAPAAVEFT

SEQ ID NO: 21 - protein, 424 aa, Zea mays
MGDASQSHSQSHSQHHGFQPQLLSFGGAGQHHAHQFTAQAASHLRPRGRGAVGAGGEAVATTSASRSRVRGGGGEAVAVQGGHIVRSTGRKDSHSKVCTARGTRDRRVRLAAHTAIQFYDVQDRLGYDRPSKAVDWLINNAKDAIDRLETTLPAWQPTANAAPPSSSWTHPDSAANSDDQAQAITVAHTAFDFPGADGFLPASLDSDTIKSFFPVAGTGGGEAEASSSAAAAQSSTMGYQSYTPDLLSRTGGHSQELRLSLQSLPDPMFHHRSHGHGHGGDGSAQQALFPGAASYSFGCGAAWAEHAQSQRVAPWNAPDPGGGSTGGYLLNVSQQAALGGQSQFFSQRGPLQSSNQPSERGWPETVEADNPMQHGGPTIGFAPGTGFSGIRIPTRIQGGEEHNGGSGNGDKPPPPPSVSSASRQ

SEQ ID NO: 22 - protein, 273 aa, Arabidopsis thaliana; identical to SEQ ID NO: 7

SEQ ID NO: 23 - protein, 273 aa, Glycine max; identical to SEQ ID NO: 7

SEQ ID NO: 24 - protein, 344 aa, Glycine max
MGMKSTGGEIVQVQGGHIVRSTGRKDRHSKVYTAKGPRDRRVRLSAHTAIQFYDVQDRLGYDRPSKAVDWLIKKAKNAIDKLAELPPWHPNPNTTAADAENNNNNNAGSSDMAIAEQSESSGYNFQLQRQLGEDHDNHHHSAFIPSPIDTDAIAFFPTTTATSSINFQTYPPDIISRTNNSTEDLGLSLHSFQDPGLIHHGQSQAGANQTPSNDQTLFSGSTQVGFEANYPRIVTWNSDASTDMNRTGFMVNSPSALLGQGGSAAYSQRGTLQSSFSPSLRPWSDIPMASSEHHHKSQQASIFGSRFLSDALPGFCIPARIQGEDEGHGVGNDRPSSASPNSHH

SEQ ID NO: 25 - protein, 458 aa, Oryza sativa
MGDAGGHSHHHQHGFQPQLLSFGGVGHHHHLHQFTAQPQPPAASHTRGRGGGGEIVPATTTPRSRGGGGGGGGEIVAVQGGHIVRSTGRKDRHSKVCTARGPRDRRVRLSAHTAIQFYDVQDRLGYDRPSKAVDWLIKNAKDAIDKLDVLPAWQPTAGGAGAGNAAAPPSSSTHPDSAENSDDQAQAITVAHTAFDFAGGGSGGTSFLPPSLDSDAIADTIKSFFPMGGTAGGEASSSTTAAQSSAMGFQSYTPDLLSRTGSQSQELRLSLQSLPDPMFHHQQHRHGGGGGGGNGTTQQALFSGAANYSFGGGAMWATEQQAQNQRMLPWNVPDPGGGGGAAYLFNVSQQAAHMQAAAAALGGHQSQFFFQRGPLQSSNQPSERGWPETVEADNQMSHHQGGLSPSVSAAIGFAAPGIGFSGFRLPARIQGDEEHNGGGGGNGDKPPPPSSVSSASHH

SEQ ID NO: 26 - protein, 462 aa, Sorghum bicolor
MGDAGQSHSHHHAFQPQLLSFGGVGQHHVHQFTAQAQAAPPAASHSRSRGGGAGGEVAVAPMSASHSRVRGGAGGGGEIVAVQGGHIVRSTGRKDRHSKVCTARGPRDRRVRLSAHTAIQFYDVQDRLGYDRPSKAVDWLIKNAKDAIDKLETLPAWQPTATGANAVAPPSSSTHPDSAENSDDEAQAITVAHTAFDFPGAAGAGGGGSGAGFLPSSLDSDSIADTIKSFFPMAGTGGGEASSSTAATQSSAMGFQSYTPDLLSRTGNHSQELRLSLQSLPDPMFHHQHQDRSQQGHGGNGSAQQALFPGAASYSFVGGGGGTMWGEQAQSQRMLPWNVPDPGGGSTGSYLFNVSQQAVSQQAAHMQAALGGQSQFFFQRGPLQSSNQPSERGWPEIVEADNPMQQQHGGGLSPTVSAIGFTPGIGFSGMRIPTRIQGDEEHNGGSGNGDKPPPSVSSASHH

SEQ ID NO: 27 - protein, 457 aa, Triticum aestivum
MGDAGQSHHHHGLHTQLLSFGGADHHMHQFTAQAQPPAASQTRARGGRGGEMVPAATPARVRGGGGGEIVAVQGGHIVRSTGRKDRHSKVCTARGPRDRRVRLSAHTAIQFYDVQDRLGYDRPSKAVDWLIKNAKDAIDNLDTLPAWQPTAVAPSASNAAAPPSSSTQPDSADNSDDQAQAITIAHSSFDFAGSGGAGGATGGISFLPPSLDSDSIADTIKSFFPMGGTAGGEPSSSTVAAHSSAMGFQGYTPDLLSRTGSQSQELRLSLQPLPDPMFHHHHHQQQQQQEQHRSHGHDGNGTGQQALFPGSYSFGGGGAMWGEQAASQRMMPWNVADPGGGSTGGYLFNVSQQAAHMQAALSGQSQFFFQRGPLQSSNQPSDRGWPETVEADDNHPMQQQQQQHQGGLNPSVSAIGFGPGVSFSGFRVPARMQGNEEHNGGNGDKPPSVSSASHHAA

SEQ ID NO: 28 - protein, 450 aa, Triticum aestivum
MGDAGQSHHHHGLQTQLLSFGGAGSHHHHMHQFTAQAQPPAASQTRARGGRGGEMVPATPARVRGGGGGEIVAVQGGHIVRSTGRKDRHSKVCTARGPRDRRVRLSAHTAIQFYDVQDRLGYDRPSKAVDWLIKNAKDAIDNLDTLPAWQPTAVAPAASNAAAPPSSSTQPDSADNSDDQAQAITIAHSSFDFAGSGGAGGATGGISFLPPSLDSDSIADTIKSFFPMGGTTGGEPSSSTVAAHSSAMGFQGYTPDLLSRTGSQSQELRLSLQPLPDPMFHHHQQQEQHRSDGHDGNGTAQQTLFPGAASYSFGGGGAMWGEQAASQRMMPWNVPDPGGGSTGGYLFNVSQQAAHMQAALSGQSQFFFQRGPLQSSNQPSDRGWPETVEADDNNPMQQHQGGLNPSVSAIGFGPGVSFSGFRVPARMQGNEEHNGGNGDKPPSVSSASHH

SEQ ID NO: 29 - protein, 454 aa, Triticum aestivum
MGDAGQSHHHHGLQTQLLSFGGAGSHHHHMHQFTAQAQAPAASQTRARGGRGGEMVPATPARVRGGGGGEIVAVQGGHIVRSTGRKDRHSKVCTARGPRDRRVRLSAHTAIQFYDVQDRLGYDRPSKAVDWLIKNAKDAIDNLDTLPAWQPTAVAPASSNAAAPPSSSTQPDSADNSDDQAQAITIAHSSFDFAGSGGAGGATGGISFLPPSLDSDSIADTIKSFFPMGGTAGGEPSSSTVAAHSSAMGFQGYTPDLLSRTGSQSQELRLSLQPLPDPMFHHHHHQQEQHRPDGHDGNGTAQQTLFPGAASYSFGGGGAMWAEQAASQRMMPWNVPDPGGGSTGGYLFNVSQQAAHMQAALSGQSQFFFQRGPLQSSNQPSDRGWPETVEADDNNPMQQQQQHQGGLNPSVSAIGFGPGVSFSGFRVPARMQGNEEHNGGNGDKPPSVSSASHH

SEQ ID NO: 30 - protein, 434 aa, Zea mays
MLLCRAAGVAAPARVLRCPLPLHSGPAACCSGTGRRISPAGKAGRRGGELSTARALPSPSLAGQIDRSPSGSRLRSLLYFRFARFPFQKEPIGLLLDRCARLDFFWEFNRRMGRDGGRVRMSRVLATGDERGMGEVAGVERAACKRPRGALDGGGSGSAAAWPACRVARAAAGGKDRHSKVVTARGLRDRRVRLSVPTAIQFYDIQDRLGVDQPSKAIEWLIRAAAAAIDGLPSLDCSFVFPHAAASPPGGAADDAELSTSETSKSSVLSLANAPVDNTAAHSQQDSHAYNNNAAAGGGAFADLILHCSANNSSKPVQQQPTLAYYATQPPNAHSASAISFETMLPHFSFLQEQPHHAIAFDRGTLQSNAAVAAPLWPTSQHACLLQRFAASPADATGLPSFFLGSGAAGASPVVPANAEPRLQLWDFKQERKT

SEQ ID NO: 31 - protein, 278 aa, Arabidopsis thaliana
MNIVSWKDANDEVAGGATTRREREVKEDQEETEVRATSGKTVIKKQPTSISSSSSSWMKSKDPRIVRVSRAFGGKDRHSKVCTLRGLRDRRVRLSVPTAIQLYDLQERLGVDQPSKAVDWLLDAAKEEIDELPPLPISPENFSIFNHHQSFLNLGQRPGQDPTQLGFKINGCVQKSTTTSREENDREKGENDVVYTNNHHVGSYGTYHNLEHHHHHHQHLSLQADYHSHQLHSLVPFPSQILVCPMTTSPTTTTIQSLFPSSSSAGSGTMETLDPRQM

SEQ ID NO: 32 - protein, 360 aa, Arabidopsis thaliana
MRSGECDEEEIQAKQERDQNQNHQVNLNHMLQQQQPSSVSSSRQWTSAFRNPRIVRVSRTFGGKDRHSKVCTVRGLRDRRIRLSVPTAIQLYDLQDRLGLSQPSKVIDWLLEAAKDDVDKLPPLQFPHGFNQMYPNLIFGNSGFGESPSSTTSTTFPGTNLGFLENWDLGGSSRTRARLTDTTTTQRESFDLDKGKWIKNDENSNQDHQGFNTNHQQQFPLTNPYNNTSAYYNLGHLQQSLDQSGNNVTVAISNVAANNNNNLNLHPPSSSAGDGSQLFFGPTPPAMSSLFPTYPSFLGASHHHHVVDGAGHLQLFSSNSNTASQQHMMPGNTSLIRPFHHLMSSNHDTDHHSSDNESDS

SEQ ID NO: 33 - protein, 478 aa, Glycine max
MEEEDEIQAQKVRRRRGSDGESTNRFHSSWHHHHHHSSRIIRVSRASGGKDRHSKVMTSKGLRDRRVRLSVTTAIQFYDLQDRLGYDQPSKAVEWLIKSASDSISELPSLNNFPDTPKSDDHEKRANTDVAAGAVDGENYENENGNNNNQSQNMSLSKSGCSSTSETSKGSGLSLSRSDIRVKARERARERTTKEKEKEKNESHHHHSNVVVPHHRHHHHHNPVSQTASFTELLTGGIPKGCDDEEPNNMMMFNKEAAAASSGRQQWCSSSSAAAAAPMDYFLGPSSSRTTTTPPHNHHHHHHQYSSSGGFSLAQIQLGHSLPEAMNHNVSAFNNNVSGENQNHSSSDPHLQQQQHFSFIPDHLMSAVVTSSSHHQPSGSDYNLNFTMSSGLAGYNRGTLQSNSPSLLPHLQRFSPLDGSTVPFFIGAAPSAAVAAPAMENNTTTTNNHHHHQFSSGFDGSRLQLYYGHSDQKGKAKN

SEQ ID NO: 34 - protein, 468 aa, Glycine max
MEEEDEILQARACKLPRRRGSDGESTNSRFHSSSWHHHNSSRIIRVSRASGGKDRHSKVMTSKGLRDRRVRLSVNTAIQFYDLQDRLGYDQPSKAVEWLIKSASDAISELPSLNNFPDTPQSDDHEKRENTADVAVGVVDGENGNGQNLSLAKSACSSNSETSKGSGLSLSRSDIRVKARERARERTTKEKEKEKEKNDESHRHHHNVVVVPHHNHNVNLVSQTASFTELLTGSDPNKGCDEANNNMMFNKAAGRLQWCSSTTPMDYFLGLSSSRTTTHNHNHLHHHQYSSSSSSGFSLAQIQLGHSLPEAMNHVSAFNNNVSGDNHNHSSSDPHLQQQHLSLIPDHLMSAVVTSSAASSHHHHHHQPSGNDYNLNSTMSSGLAGYNRGTLQSNSPSLLPHSQRFSPIDGSTVPFFIGAASSAAAVAAPAMENNNNNNHHQHQFSSVFDGSRLQLYYGHSDQKGKAKN

SEQ ID NO: 35 - protein, 532 aa, Glycine max
MEEDEIQACKFPRVGNGRSSDQYQEEEEEGGDLRLRKGVGVGGGGGGGGSDASTNHFHQSSWHHSSRIIRVSRASGGKDRHSKVMTSKGLRDRRVRLSVTTAIQFYDLQDRLGYDQPSKAVEWLIKAASEAISELPSLNNPFPDTPKQLSDKKRPTSGGGQQQQQIQQGHFDDADGDTSYPQNQSQNLSLSKSACSSTSETSKGSGLSLSRSEIRVNRVKARERARERAAKEKEKEKEKEKEKEKESESSIAHHHHHHHVNNNNHMSHTASFTELLTGGIGSTVTVNPNTTTTTTTTTTSPNGSSVHQIHDGHDHETNLFNKGRQQQQQHHWSQTVTPMDYFSTGLLVGPSSSSARTQQHQHQSSSAHFQLGHAHALPISPFSGENHSEQLQHFSFMPDHLNNIPAHHVVTSSSASHQPNGGDNNYNLNFSISTSTGLAGYNNRGTLQSNSPSSSFLPHHLQRFQPLDGSSNLPFFIGAAAPSSAPPTMDNNNNNNSNNHHHLQFSPVFDGRLQLCYGDGTRHSDHKGKGKN

SEQ ID NO: 36 - protein, 511 aa, Glycine max
MEEDEIQACKFPRVGNGRSSDQYQEEEEEGDLRRKGVGGGGGSDASTNHFHQSSWHYSSRIIRVSRASGGKDRHSKVMTSKGLRDRRVRLSVTTAIQFYDLQDRLGYDQPSKAVEWLIKAASEAISELPSLNNPFPDTPKQPSDEKRPTSGGGQQQQQQGFDDADGDTNYQQNQSQNLSLSKSACSSTSETSKGSGLSLSRSEIRVNRVKARERARERAAKEKEKEKEKESDFNIAHHHHHHNVNNMSHTASFTELLTGGIGSTVTVNPNTTTTTTTTATSPNGSSVHQIHEGHDHEANLFNKGSQQQQQQQHHHWSQTVTPMDYFSAGLLVGPSSSASTQQHQSSGQFQLGHGHAHAHALSISPFSGENHSEQMQHFSFMPDHLNNMPSSAASASQHSEGDNNNYNLNFSISSSGLAGYNRGTLQSNSPSSSFLPLLQRFQPLVDGSSNLPFFIGAAPPSSAPPTMDNNNSNNSNNHHHLQFSPIFDGRLQLCYGDGTRQHSDHKGKGKN

SEQ ID NO: 37 - protein, 266 aa, Gossypium raimondii
MISSSREKSCFEAKQEGDGGKVSSSRQWQGFRNPRIVRVSRSFGGKDRHSKVCTIRGLRDRRIRLSVPTAIQLYDLQDRLGLNQPSKVIDWLLEATKDDVDKLPPLEFHHSLMPNPCQSSPFTPLMDPNLMFMKDYEGETSMQVEREITDMKGKWIKEQEKLIFPVTNHGSSLPGGFMFNTGTNSMPLNTYNHHWNPSQFTNHGFHHHHQPENYFHGAIANTYPPLPSCPPPSTTPSHFPTFPWYGTNTNQDAAGDQDNSKLQFFT

SEQ ID NO: 38 - protein, 285 aa, Gossypium raimondii
MISSSKNTDFPTRQDEDDDKRNETNKGPSSSSSSSQWLRLKDPRIVRVSRAFGGKDRHSKVYTIRGLRDRRVRLSVPTAIQLYDLQDRLGLNQPSKVVDWLLNAAKHEIDELPPLPMPPPPANFPISDAFWRTKSKEVTRDVIDDKDDKHRRNEGDDDDDHGTQHVSSGSYYHFEPSNFPLSSHLVSHGFAVHAPQVDNFNVVAAPLPSALSLCPLPPPQPGGGAPPLFPPHVVDPRQQVNHFQMLSSGAQQNLLLNSLNFPPITQSFRPFQLMPPRFPHNQPDK

SEQ ID NO: 39 - protein, 309 aa, Gossypium raimondii
MIPSSEEAGSPIQEGKEDESSNKVRKGASTSSPLLRLKDPRIVRVSRAFGGKDRHSKVFTVRGLRDRRVRLSVPTAIQLYDLQDRLGLNQPSKVVDWLLNEAKHEIEELPPLPIPQGNFSLNPQMPLNASQQPNKQDSTGGFCRAKSKDIVRESSKGSKRDEEASEGHQVQTDYGLQRSNHSPMPALSNNAVSMPMPMPMPYGSYYHFEPSNFPLPHLGSHGFVAPQTEEVHNFNVVPLSSTLSLSSGSQNHFQMLSSGAQNPFANPPQYSITQPDLRSFHLSIAPRLLPHSLNINGSQPGKEDEFPSK

SEQ ID NO: 40 - protein, 357 aa, Oryza sativa
MEAAVGDGEGGGGGGGRGKRGRGGGGGEMVEAVWGQTGSTASRIYRVRATGGKDRHSKVYTAKGIRDRRVRLSVATAIQFYDLQDRLGFDQPSKAIEWLINAASPAIDTLPSLDPAAFAAIPHAAADAAPTRRRSQQQQQQLSNKSGCSSTSETSKGSDKEVTVASAPAQAASFTELLIAGVAASSAGGGAIGNGADCVGIAHPGKGGAEGASTYGFSAASSFGDAPPIGMVPAPPFNFSAPGADMAAHYSLAQDQLAAPPPPAGGDYNLNFSMSSGFLGANRGTLQSNSPSNMSGHHHHHHQQQLQRLDGSTISFLLGHAAAAAHPAASEGQITSTAALQLWDGFRHSGMKEKSKN

SEQ ID NO: 41 - protein, 445 aa, Oryza sativa
MELAGSNNKRGRVRGPNDDDDDAGEPDAKRHHHQLLLPWPQQQQQQHPASRIYRVSRASGGKDRHSKVYTAKGIRDRRVRLSVSTAIQFYDLQDRLGYDQPSKAIEWLIKAAAAAIDKLPSLDTASFPTNPASSAAVAAAAAPPLPHAEREQQQQLTKSGCSSTSETSKGSVLSLSRSESRVKARERARERSSAAAAAASKDAGDDAATPTAPTAAPASSQAASFTELLTGMAAANASPADHKQQQAWQPMTVAAATADYIGFAAAAAPHTQPRKSAAGHHSAMPHTFASPAPHLANITPIAMAPAQHFTLTPAAAEHHAEMTHYSFDHFMPVHAAAAAAAAASTPAGGDYNLNFSMSSGLVGVHSRGTLQSNSQSHLSSHHHHHHQQQQQQQQLQRLSAPLDAPNIPFLFSPAAAPTAADTQFAAALQLWDGFRHADIKEKGKH

SEQ ID NO: 42 - protein, 302 aa, Sorghum bicolor
MEEVAVAAGMERAAGCKRPRGALDGGGSASAAAWRTCRVARAAAGGKDRHSKVVTARGLRDRRVRLSVPTAIQFYDIQDRLGVDQPSKAIEWLIRAAAAAIDGLPSLDCSFALPTAAGAASPPGAADDAEVSTSDTSKSSVLSLANASADNAAAQQASHAYNGNAAGGGGAFAELILHCSPNDSKPVQQQPTLAYYAAQPPSAHAASALSSFETMPHFSFLQEQPHPAVAFDRGTLQSNAAVAAPLWPTSQQACLLQRFAASPADATGLPFFLGGSAAASPMVPDNSEPRLQLWDFKQERKT

SEQ ID NO: 43 - protein, 297 aa, Triticum aestivum
MEEADKDRNSCKRLRAVGGGDSADAWRTFRVARAAAGGKDRHSKVVTSRGLRDRRIRLSVQTAIQFYDIQDRLGVDQPSKAIEWLIQAAATAIDGLPSLDCSFALPVASPAAEDAVEVSTSETSKGSVLSLANGPADNDNAAHQANAYNGGSANGTFAELLHCSNAADKPMQHQQQQPTLAYYAAPGSHIAPMSFEMIPQLTFSQDQQHRHHHHHHHHHASVVFDRSTLQSNTVSTPQWPPSQHPFLMQRFSAAPAEPSPMFPFFLGGNSAAAAAPAAANAPERRLQLWDFKEERKT

SEQ ID NO: 44 - protein, 300 aa, Triticum aestivum
MEEADKDRNSCKRLRAVGGGGDSADAWRSFRVARAAAGGKDRHSKVVTSRGLRDRRIRLSVQTAIQFYDIQDRLGVDQPSKAIEWLIQAAATAIDGLPSLDCSFAFPVASPAAEDTAEVSTSETSKGSVLSLANGPADNDNAAHQASAYNGGSANGTFAGLLHCSNAADKPMQHQHQQQQPTLAYYAAPGSHIAPMSFEMIPQLTFSQDQQHCHHHHHHHHASVSFDRSTLQSNTVSTPQWPPSQHPFLMQRFSAAPAEPSPMFPFFLGGNNAAAAAAPAAANAPERRLQLWDFKEERKT

SEQ ID NO: 45 - protein, 295 aa, Triticum aestivum
MEEADKDRNSCKRLRAVGGGGDSADAWRTFRVARAAAGGKDRHSKVVTSRGLRDRRIRLSVQTAIQFYDIQDRLGVDQPSKAIEWLIQAAATAIDGLPSLDCSFALPVASPAAEDAAEVSTSETSKGSVLSLANGPADNDNATHQANAYNGGSANGTFAELLHCSNAADKPMQHQHQQPTLAYYAAPGSHIAPMSFEMIPQLTFSQDQQHRHHHHHHASVAFDRSTLQSNTVSTPQWPPSQHPFLMQRFSAAPAEPSPMFPFFLGGNSASAAAPAAANAPERRLQLWDFKEERKT

SEQ ID NO: 46 - RNA, 23 nt, synthetic construct
  source 1..23: mol_type = other RNA
tcgaggagggagctgtccggtgg

SEQ ID NO: 47 - RNA, 23 nt, synthetic construct
  source 1..23: mol_type = other RNA
ggagggagccgtccggtgccggg

SEQ ID NO: 48 - RNA, 23 nt, synthetic construct
  source 1..23: mol_type = other RNA
aggcaggggtagttcagcgccgg

SEQ ID NO: 49 - RNA, 23 nt, synthetic construct
  source 1..23: mol_type = other RNA
tatatgcagggacgtggtgcagg

SEQ ID NO: 50 - RNA, 23 nt, synthetic construct
  source 1..23: mol_type = other RNA
ggcgcgatgtcaaagctggccgg

SEQ ID NO: 51 - RNA, 23 nt, synthetic construct
  source 1..23: mol_type = other RNA
cggacgggtcagccggcacctgg

SEQ ID NO: 52 - RNA, 23 nt, synthetic construct
  source 1..23: mol_type = other RNA
tgacgggacaggggaggccttgg

SEQ ID NO: 53 - RNA, 23 nt, synthetic construct
  source 1..23: mol_type = other RNA
cgacgagatcttcacggtgttgg

SEQ ID NO: 54 - RNA, 23 nt, synthetic construct
  source 1..23: mol_type = other RNA
ccgcgccggccgggccgttacgg

SEQ ID NO: 55 - RNA, 23 nt, synthetic construct
  source 1..23: mol_type = other RNA
acggcggcccaatgcccgtgagg

SEQ ID NO: 56 - RNA, 23 nt, synthetic construct
  source 1..23: mol_type = other RNA
gtacacctctgtgagtgagtcgg

SEQ ID NO: 57 - RNA, 23 nt, synthetic construct
  source 1..23: mol_type = other RNA
gataactcccatgttgcaatagg

SEQ ID NO: 58 - RNA, 23 nt, synthetic construct
  source 1..23: mol_type = other RNA
aggccccgcacgtgcttgagcgg

SEQ ID NO: 59 - RNA, 23 nt, synthetic construct
  source 1..23: mol_type = other RNA
agctcagctgccccgagaccagg

SEQ ID NO: 60 - RNA, 23 nt, synthetic construct
  source 1..23: mol_type = other RNA
ttgggcggagcaacttgcagcgg

SEQ ID NO: 61 - RNA, 23 nt, synthetic construct
  source 1..23: mol_type = other RNA
atagcactcttccatctgactgg

SEQ ID NO: 62 - RNA, 23 nt, synthetic construct
  source 1..23: mol_type = other RNA
tgagacgctcaagcacggtctgg

SEQ ID NO: 63 - RNA, 23 nt, synthetic construct
  source 1..23: mol_type = other RNA
cttgagcgaggtgagcttcacgg

SEQ ID NO: 64 - RNA, 23 nt, synthetic construct
  source 1..23: mol_type = other RNA
tgttgtggctgttaccctttggg

SEQ ID NO: 65 - RNA, 23 nt, synthetic construct
  source 1..23: mol_type = other RNA
ttcctggaccgcgtgagcgccgg

SEQ ID NO: 66 - RNA, 23 nt, synthetic construct
  source 1..23: mol_type = other RNA
cttcctgaaccgggtcaggatgg

SEQ ID NO: 67 - RNA, 23 nt, synthetic construct
  source 1..23: mol_type = other RNA
tgttcatggcagctctaggcggg

SEQ ID NO: 68 - RNA, 23 nt, synthetic construct
  source 1..23: mol_type = other RNA
gaactcggacctgttcatggcgg

SEQ ID NO: 69 - RNA, 23 nt, synthetic construct
  source 1..23: mol_type = other RNA
cacccaatgctgcgtagaacagg

SEQ ID NO: 70 - RNA, 23 nt, synthetic construct
  source 1..23: mol_type = other RNA
catggaggatatccacgcaccgg

SEQ ID NO: 71 - RNA, 23 nt, synthetic construct
  source 1..23: mol_type = other RNA
aggtcattgactcagtccactgg

SEQ ID NO: 72 - RNA, 23 nt, synthetic construct
  source 1..23: mol_type = other RNA
ctcgcggtctgggccgctcccgg

SEQ ID NO: 73 - RNA, 23 nt, synthetic construct
  source 1..23: mol_type = other RNA
tgtcgtcaccgcgagcaactcgg

SEQ ID NO: 74 - RNA, 23 nt, synthetic construct
  source 1..23: mol_type = other RNA
cgcagaagctgtgctcgcgacgg

SEQ ID NO: 75 - RNA, 23 nt, synthetic construct
  source 1..23: mol_type = other RNA
cttacgcctctgcgaatcgccgg

SEQ ID NO: 76 - RNA, 23 nt, synthetic construct
  source 1..23: mol_type = other RNA
ctcctcacggacgccacctgcgg

SEQ ID NO: 77 - RNA, 23 nt, synthetic construct
  source 1..23: mol_type = other RNA
cccgacctcctcgtggtcgccgg

SEQ ID NO: 78 - RNA, 23 nt, synthetic construct
  source 1..23: mol_type = other RNA
ccgcacgctcgcgctgcagaggg

SEQ ID NO: 79 - RNA, 23 nt, synthetic construct
  source 1..23: mol_type = other RNA
cggtatgaaccggcagctcgggg

SEQ ID NO: 80 - RNA, 23 nt, synthetic construct
  source 1..23: mol_type = other RNA
ggctcgcccatgcacttcttcgg

SEQ ID NO: 81 - RNA, 23 nt, synthetic construct
  source 1..23: mol_type = other RNA
gacactcgtgccagaacctgtgg

SEQ ID NO: 82 - RNA, 23 nt, synthetic construct
  source 1..23: mol_type = other RNA
ttctttgttccattgagaggagg

SEQ ID NO: 83 - RNA, 23 nt, synthetic construct
  source 1..23: mol_type = other RNA
ggacctccggatcctcgaccagg

SEQ ID NO: 84 - RNA, 23 nt, synthetic construct
  source 1..23: mol_type = other RNA
ttctgtcctgttcgcaggagagg

SEQ ID NO: 85 - RNA, 23 nt, synthetic construct
  source 1..23: mol_type = other RNA
catccgtccctgacggtgctggg

SEQ ID NO: 86 - RNA, 23 nt, synthetic construct
  source 1..23: mol_type = other RNA
cactggacatatgaacaaaccgg

SEQ ID NO: 87 - RNA, 23 nt, synthetic construct
  source 1..23: mol_type = other RNA
tgcccaaccatgaggtcctgcgg

SEQ ID NO: 88 - RNA, 23 nt, synthetic construct
  source 1..23: mol_type = other RNA
ccagttcccgccgcaagggcagg

SEQ ID NO: 89 - RNA, 23 nt, synthetic construct
  source 1..23: mol_type = other RNA
gtaccggtccaagactcgccggg

SEQ ID NO: 90 - RNA, 23 nt, synthetic construct
  source 1..23: mol_type = other RNA
aggcgcgcacgggctgtcgacgg

SEQ ID NO: 91 - RNA, 23 nt, synthetic construct
  source 1..23: mol_type = other RNA
ctcgaaggatatggccgacgcgg

SEQ ID NO: 92 - RNA, 23 nt, synthetic construct
  source 1..23: mol_type = other RNA
cagcacttcggcttggccagtgg

SEQ ID NO: 93 - RNA, 23 nt, synthetic construct
  source 1..23: mol_type = other RNA
cagctcctggctgtgtccgccgg

(19) United States
(12) Patent Application Publication
(10) Pub. No.: US 2026/0286377 A1
(43) Pub. Date: Sep. 24, 2026

(54) Allelic Combinations in Crops for Yield Increase

(71) Applicants: VIB VZW, Gent (BE); Universiteit Gent, Gent (BE)

(72) Inventors: Laurens Pauwels, Gent (BE); Dirk Gustaaf Inzé, Gent (BE); Hilde Nelissen, Gent (BE); Christian Lorenzo, 9052 (BE); Lennert Impens, Gent (BE)

(21) Appl. No.: 19/477,030

(22) PCT Filed: Apr. 19, 2025
(86) PCT No.: PCT/EP2024/060715

§ 371 Date: Oct. 20, 2025

(30) Foreign Application Priority Data
Apr. 21, 2023 (EP) 23169181.7
Jan. 12, 2024 (EP) 24151497.5

(51) Int. Cl. C12N 15/82

(52) U.S. Cl. CPC C12N 15/8261

(57) ABSTRACT

The present invention relates to cultivated crops which have increased yield as well as compositions and methods for increasing yield.

### CROSS REFERENCE TO RELATED APPLICATIONS

This application is a national phase entry under 35 U.S.C. § 371 of International Patent Application PCT/EP2024/060715, filed Apr. 19, 2024, designating the United States of America and published in English as International Patent Publication WO2024/218295 on Oct. 24, 2024, which claims the benefit under Article 8 of the Patent Cooperation Treaty to European Patent Application Serial No. 23169181.73, filed Apr. 21, 2023 and to European Patent Application Serial No. 24151497.5 filed Jan. 12, 2024, the entireties of which are hereby incorporated by reference.

### INCORPORATION BY REFERENCE

The ST.26 XML Sequence listing named “807_PCT.xml”, created on Apr. 19, 2024, and having a size of 118,784, is hereby incorporated herein by this reference in its entirety.

### FIELD OF THE INVENTION

The present invention relates to cultivated crops which have increased yield as well as compositions and methods for increasing yield.

### INTRODUCTION TO THE INVENTION

Gene editing is the preferred tool for the creation of novel alleles for plant genetic research and breeding (Zhu et al., 2020). Subsequent phenotypic analysis is used to associate genes to (molecular) functions and traits such as nutritional quality, yield, and resistance to (a) biotic stress (H.-J. Liu et al., 2020; Lorenzo et al., 2023). Because drought, heat, salt stress and yield are likely to become more important due to climate change, plant responses to these abiotic stresses are intensely studied to improve agricultural production and contribute to the sustainability of the global food system (Verslues et al., 2023). Many of these plant traits are controlled by a complex interaction between many different genes and require the identification of the right combinations of alleles to have pronounced and desired effects (Vanhaeren et al., 2014; Vanhaeren et al., 2016; Sun et al., 2017). Having the right combinations of alleles is also valuable in overcoming genetic redundancy, which is common in plants (Corcoran et al., 2022). Hence, the ability to stack multiple gene edits in the same plant is crucial for the engineering of complex traits. Multiplexing, i.e. the simultaneous targeting of many genes, is one of the major advantages of the CRISPR/Cas9 gene editing system. Delivery of the Cas9 protein can be combined with a single T-DNA containing multiple guide RNAs (gRNAs), only differing in the spacer sequence and targeting a variety of genes, including multiple members of gene families (Lorenzo et al., 2023).

In the present invention we disclose combinations of gene disruptions in growth regulating factors which lead to an increased plant growth.

### FIGURES

The patent or application file contains at least one drawing executed in color. Copies of this patent or patent application publication with color drawing(s) will be provided by the Office upon request and payment of the necessary fee.

FIGS. 1A-1D Efficient haploid induction and haploid doubling using B104. (FIG. 1A) Number of embryos obtained per cross between B104 background (female) and RWS-GFP (male) (n=38 independent pollinations). (FIG. 1B) Haploid induction rate (HIR). Percentage of embryos scored as haploid based on the absence of GFP from a cross between B104 background and RWS-GFP (n=38). (FIG. 1C) Percentage of haploid plants in each independent experiment surviving the embryo rescue doubling process (n=30). (FIG. 1D) Haploid doubling rate. Percentage of plants in each experiment scored either as doubled haploid (DH), mixoploid, or haploid based on flow cytometry analysis of leaf 3 or 4, calculated per treated haploid embryo (n=28). Boxplots with jittered data points; center lines show the medians; box limits indicate the 25th and 75th percentiles; whiskers extend 1.5 times the interquartile range from the 25th and 75th percentiles, and the mean is indicated as a cross.

FIGS. 2A and 2B. Combining multiplex gene editing and doubled haploid breeding in maize. (FIG. 2A) Overview of the HI-BREEDIT strategy. For simplicity, six GE target loci are shown to represent a genotype, stacked bars in shades of green represent different edited alleles, and white bars indicate reference alleles. Multiplex edited TO plants are crossed with wild-type (WT) B104 plants resulting in BC1 plants that are either heterozygous or wild-type at each locus and can be selected for the absence of the EDITOR T-DNA to avoid further editing. BC1 plants are pollinated using a haploid inducer line carrying a GFP transgene (RWS-GFP). Two weeks after pollination, embryos are isolated, haploid embryos are selected for the absence of GFP fluorescence (GFP-), and incubated on a medium containing colchicine. Colchicine-treated embryos are germinated in vitro and chromosome doubling is confirmed by flow cytometry. Every haploid embryo and DH plant will show a random combination of edited loci (only one genotype is shown as an example). Doubled haploids (DH0) are self-pollinated to obtain genetically identical homozygous DH1 seeds. (FIG. 2B) Diagrams of the EDITOR and SCRIPT T-DNAs. RB, right border; pZmUBI, maize UBIQUITIN-1 promoter; tNOS, Agrobacterium NOPALINE SYNTHASE terminator; p35S, double Cauliflower mosaic virus 35S promoter; HYG, HYGROMYCINE RESISTANCE; BAR, BIALAPHOS RESISTANCE, LB, left border. In the SCRIPT T-DNA, 12 gRNAs are alternately expressed by either an OsU3 or TaU3 promoter.

FIG. 3. Combining haploid induction and multiplex gene editing with SCRIPT 4. Maize B104 immature embryos heterozygous for the EDITOR T-DNA were supertransformed with the SCRIPT 4 gRNA construct, targeting the 12 genes listed on top. Experimentally obtained haplotypes for a representative subset of plants are shown for each generation. Colored, horizontally stacked bars each indicate different mutant out-of-frame alleles per target locus, white bars indicate wild-type (reference) alleles, light gray bars indicate in-frame mutant alleles, and colored bar lengths are proportional to the fraction of sequence reads per locus containing the allele. Bar lengths <50% are indicative of mosaicism.

FIG. 4. Phenotypic analysis of SCRIPT 4 DH1 populations. Genotypes and corresponding phenotypes observed in DH1 SCRIPT 4 populations homozygous for various combinations of out-of-frame alleles (green squares) and in-frame mutated alleles (gray squares); the size of the indel (in bp) is indicated in the squares. White squares indicate that the wild-type reference (REF) allele was identified by genotyping. Each row represents an independent DH1 population of plants. Boxplots with jittered data points on the right display measurements of pseudo leaf 3 area (PLA3) for edited plants compared with non-edited control plants (wild-type B104 and two wild-type doubled haploids (HIC01 and HIC03)). DH1 lines are sorted from lowest to highest mean PLA3. 24 to 29 seeds were sown for each DH1 line; n, number of germinated plants phenotyped. The compact letter display shows the result of the pairwise comparisons of the Wilcoxon rank sum test (significance level of 5% with Holm correction).

FIGS. 5A and 5B. Combining haploid induction and multiplex gene editing with inter-script populations. Experimentally obtained haplotypes for a representative subset of plants of (FIG. 5A) S2xS4 and (FIG. 5B) S4xS3 are shown for each generation. Colored, horizontally stacked bars each indicate different mutant out-of-frame alleles per locus, white bars indicate wild-type (reference) alleles, light gray bars indicate in-frame mutant alleles, and colored bar lengths are proportional to the fraction of sequence reads per locus containing the allele. Absent bars indicate missing data due to low-quality sequencing. Gene locus names are indicated above each column, as well as their respective SCRIPT construct.

FIGS. 6A and 6B. Phenotypic analysis of DH1 and DH2 populations. (FIG. 6A) Observed genotypes and corresponding phenotypes in DH1 and DH2 for four different homozygous edited genotypes and the non-edited control HIC01. Out-of-frame mutated alleles (green squares), in-frame mutated alleles (gray squares) and reference alleles (white squares), the size of the indel (in bp) is indicated in the squares. On the left, each row represents the genotype of a population (DH1 or DH2), on the right, corresponding boxplots with jittered data points display measurements of pseudo leaf 3 area (PLA3). 24 to 35 seeds of each population were sown; n, number of plants phenotyped. The compact letter display shows the result of the pairwise comparisons of the Wilcoxon rank sum test (significance level of 5% with Holm correction). (FIG. 6B) Representative seedlings at V3 stage for HIC01 and the septuple mutant Pop25, one plant of each generation (DH1 and DH2).

FIGS. 7A and 7B. Phenotypic analysis of SCRIPT 4 DH1 populations. Genotypes and corresponding phenotypes observed in DH1 SCRIPT 4 populations homozygous for various combinations of out-of-frame alleles (green squares) and in-frame mutated alleles (gray squares); the size of the indel (in bp) is indicated in the squares. White squares indicate that the wild-type reference (REF) allele was identified by genotyping. Each row represents an independent DH1 population of plants. Boxplots with jittered data points on the right display measurements of (FIG. 7A) final leaf 3 length (FLL3) and (FIG. 7B) final leaf width (FLW3) for edited plants compared with non-edited control plants (wild-type B104 and two wild-type doubled haploids (HIC01 and HIC03)). DH1 lines are sorted from lowest to highest mean FLL3 (FIG. 7A) or FLW3 (FIG. 7B). 24 to 29 seeds were sown for each DH1 line; n, number of germinated plants phenotyped. The compact letter display shows the result of the pairwise comparisons of the Wilcoxon rank sum test (significance level of 5% with Holm correction).

FIGS. 8A and 8B: Pseudo leaf area for the single gene disrupted plants TCP42 and GRF10 and the double combination (TCP42xGRF10). The double combination has more than 15% increase in pseudo leaf area (FIG. 8A). Pseudo leaf area for the single gene disrupted plants TCP9 and GRF4 and the double combination (TCP9 x GRF4). The double combination has more than 11% increase in pseudo leaf area (FIG. 8B).

FIG. 9. Phenotypic screen of PLA3 in inter-script DH1 populations. Genotypes and corresponding phenotypes observed in DH1 inter-script populations homozygous for various combinations of out-of-frame alleles (green squares) and in-frame mutated alleles (gray squares). White squares indicate that the wild-type reference (REF) allele was identified by genotyping. Each row represents an independent DH1 population of plants. Boxplots with jittered data points on the right display measurements of pseudo leaf 3 area (PLA3) for edited plants compared with non-edited control plants (wild-type B104, EDITOR 1 without SCRIPT (ED1) and a wild-type doubled haploid (HIC01)). DH1 lines are sorted from lowest to highest mean PLA3. Eight to twelve DH1 seeds were sown for each DH1 line and 30 seeds for three control lines; n, number of germinated plants phenotyped. The compact letter display shows the result of the pairwise comparisons of the Wilcoxon rank sum test (significance level of 5% with Holm correction). Absent squares for TCP42 indicate missing data due to low-quality amplicons. The FLL3 and FLW3 components of PLA3 are plotted individually in FIG. 10.

FIGS. 10A and 10B. Phenotypic screen of FLL3 and FLW3 in inter-script DH1 populations. Genotypes and corresponding phenotypes observed in DH1 inter-script populations homozygous for various combinations of out-of-frame alleles (green squares) and in-frame mutated alleles (grey squares). Each row represents an independent DH1 population of plants. Boxplots with jittered data points on the right display measurements of (FIG. 10A) final leaf 3 length (FLL3) and (FIG. 10A) final leaf width (FLW3) for edited plants compared to non-edited control plants (wild-type B104, EDITOR 1 without SCRIPT (ED1) and a wild-type doubled haploid (HIC01)). DH1 lines are sorted from lowest to highest mean FLL3 (FIG. 10A) or FLW3 (FIG. 10B). Eight to twelve DH1 seeds were sown for each DH1 line and 30 seeds for three control lines; n, number of germinated plants phenotyped. The compact letter display shows the result of the pairwise comparisons of the Wilcoxon rank sum test (significance level of 5% with Holm correction). Absent squares for TCP42 indicate missing data due to low-quality amplicons.

FIGS. 11A and 11B. Phenotypic analysis of DH1 and DH2 populations. Genotypes and corresponding phenotypes observed in DH1 and DH2 for four different homozygous edited lines and non-edited control lines (HIC01). Out-of-frame mutated alleles (green squares), in-frame mutated alleles (grey squares) and reference alleles (white squares), the size of the indel (in bp) is indicated in the squares. On the left, each row represents the genotype of a population (DH1 or DH2), on the right, corresponding boxplots with jittered data points display measurements of (FIG. 11A) final leaf 3 length (FLL3) and (FIG. 11B) final leaf width (FLW3). 24-35 seeds of each population were sown; n, number of germinated plants phenotyped. The compact letter display shows the result of the pairwise comparisons of the Wilcoxon rank sum test (significance level of 5% with Holm correction).

FIGS. 12A-12C. Fresh biomass parameters of adult plants. DH2 seeds of the a wild-type doubled haploid (HIC3), tcp42, grf10 single mutants, and grf10 tcp42 double mutant were grown to maturity in a greenhouse and phenotypes of each plant were recorded at the day of silking. For fresh biomass parameters, aboveground plants parts were harvested and the fresh stem weight (FIG. 12A), fresh leaf weight (FIG. 12B) and total biomass of leaves and stems (FIG. 12C) determined. An ANOVA test was conducted between the HIC03 control and DH2 plants followed by a Tukey's post hoc test (n=15). Significant differences are displayed with P-values compared to wt (HIC3) summarized as follows: *P<0.05, ** P<0.01, *** P<1e-3, **** P<1e-4. The middle line represents the means of each distribution and the error bars the standard error of the mean (SEM).

FIGS. 13A-13C. Dry biomass parameters of adult plants. DH2 seeds of the a wild-type doubled haploid (HIC3), tcp42, grf10 single mutants, and grf10 tcp42 double mutant were grown to maturity in a greenhouse and phenotypes of each plant were recorded at the day of silking. For dry biomass parameters, aboveground plants parts were harvested, dried and the dry stem weight (FIG. 13A), dry leaf weight (FIG. 13B) and total biomass (FIG. 13C) determined. An ANOVA test was conducted between the HIC03 control and DH2 plants followed by a Tukey's post hoc test (n=15). Significant differences are displayed with P-values compared to wt (HIC3) summarized as follows: * P<0.05, ** P<0.01, *** P<1e-3, **** P<1e-4. The middle line represents the means of each distribution and the error bars the standard error of the mean (SEM).

FIGS. 14A-14C. Height (FIG. 14A) and leaf parameters (FIG. 14B) (FIG. 14C) of adult plants. DH2 seeds of the a wild-type doubled haploid (HIC3), tcp42, grf10 single mutants, and grf10 tcp42 double mutant were grown to maturity in a greenhouse and phenotypes of each plant were recorded at the day of silking. Height of the plants was determined by measuring the distance from the crown to the highest collar. For the leaf under the primary developing ear (cob leaf) width and length was determined. An ANOVA test was conducted between the HIC3 control and DH2 plants followed by a Tukey's post hoc test (n=15). Significant differences are displayed with P-values compared to wt (HIC3) summarized as follows: *P<0.05, ** P<0.01, *** P<1e-3, **** P<1e-4. The middle line represents the means of each distribution and the error bars the standard error of the mean (SEM).

FIGS. 15A-15C. Stem parameters of adult plants. DH2 seeds of the a wild-type doubled haploid (HIC3), tcp42, grf10 single mutants, and grf10 tcp42 double mutant were grown to maturity in a greenhouse and phenotypes of each plant were recorded at the day of silking. Maximal (FIG. 15A) and minimal (FIG. 15B) stem diameter was determined at the primary cob node; (FIG. 15C) representative pictures of stems. A pairwise Student's t-test was conducted between the HIC03 control and DH2 plants (n=15). An ANOVA test was conducted between the HIC3 control and DH2 plants followed by a Tukey's post hoc test (n=15). Significant differences are displayed with P-values compared to wt (HIC3) summarized as follows: *P<0.05, ** P<0.01, *** P<1e-3, **** P<1e-4. The middle line represents the means of each distribution and the error bars the standard error of the mean (SEM).

FIGS. 16A-16D. Seed yield potential of adult plants. DH2 seeds of the a wild-type doubled haploid (HIC3), tcp42, grf10 single mutants, and grf10 tcp42 double mutant were grown to maturity in a greenhouse and phenotypes of each plant were recorded at the day of silking. Seed yield potential was determined by measuring the number of rows (FIG. 16A) and the number spikelets per row (FIG. 16B) of the unpollinated ears. The total number of spikelets (FIG. 16C) was estimated by multiplying the number of spikelets per row for each ear with the number of rows; (FIG. 16D) Representative immature cobs of wt (HIC3), single and double mutants collected simultaneously at the day of silking. Violin plots dashed lines represent local high frequent values in the distribution. An ANOVA test was conducted between the HIC3 control and DH2 plants followed by a Tukey's post hoc test (n=15). Significant differences are displayed with P-values compared to wt (HIC3) summarized as follows: * P<0.05, ** P<0.01, *** P<1e-3, **** P<1e-4. The middle line represents the means of each distribution and the error bars the standard error of the mean (SEM).

### DESCRIPTION OF THE INVENTION

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone)

As used herein, the terms “Cpf1” and “Cas12a” are used interchangeably to refer to the same RNA dependent DNA endonuclease (RdDe).

As used herein, the phrase “elite crop plant” refers to a plant which has undergone breeding to provide one or more trait improvements. Elite crop plant lines include plants which are an essentially homozygous, e.g., inbred or doubled haploid. Elite crop plants can include inbred lines used as is or used as pollen donors or pollen recipients in hybrid seed production (e.g., used to produce F1 plants). Elite crop plants can include inbred lines which are selfed to produce non-hybrid cultivars or varieties or to produce (e.g., bulk up) pollen donor or recipient lines for hybrid seed production. Elite crop plants can include hybrid F1 progeny of a cross between two distinct elite inbred or doubled haploid plant lines.

As used herein, the phrase “endogenous gene” refers to the native form of a gene unit in its natural location in the genome of an organism.

As used herein, the term “expression” refers to the production of a functional end-product (e.g., an mRNA, guide RNA, or a protein) in either precursor or mature form.

As used herein, the term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell and includes reference to the transient provision of a nucleic acid or protein to the cell. Introduced includes reference to stable or transient transformation methods. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., nuclear chromosome, plasmid, plastid, chloroplast, or mitochondrial DNA), converted into an autonomous replicon, or transiently expressed (e.g., transfected mRNA).

As used herein, a “loss-of-function allele” can include an amorphic allele or a hypomorphic allele of a gene.

As used herein, the term “plant” includes a whole plant and any descendant, cell, tissue, or part of a plant. The term “plant parts” include any part(s) of a plant, including, for example and without limitation: seed (including mature seed and immature seed); a plant cutting; a plant cell; a plant cell culture; or a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. Regenerable cells in a plant cell or tissue culture may be embryos, protoplasts, meristematic cells, callus, pollen, leaves, anthers, roots, root tips, silk, flowers, kernels, ears, cobs, husks, or stalks. In contrast, some plant cells are not capable of being regenerated to produce plants and are referred to herein as “non-regenerable” plant cells.

The term “selecting”, as used herein, refers to a process of picking out a certain individual plant from a group of individuals, usually based on a certain identity, trait, characteristic, and/or molecular marker of that individual.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

The present disclosure provides for a cultivated crop plant selected from
i) a crop plant comprising a loss-of-function allele of a first endogenous gene encoding for a polypeptide comprising SEQ ID NO: 1 and 2 and comprising a loss-of-function allele of a second endogenous gene encoding for a polypeptide comprising SEQ ID NO: 3 and 4 or
ii) a crop plant comprising a loss-of-function allele of a first endogenous gene encoding for a polypeptide comprising SEQ ID NO: 1 and 2 and comprising a loss-of-function allele of a second endogenous gene encoding for a polypeptide comprising SEQ ID NO: 5 and 6.

In a particular embodiment the cultivated crop plant is homozygous for the first and second loss-of-function alleles of the endogenous genes.

In another particular embodiment the cultivated crop plant which comprises a loss-of-function allele comprises a frameshift mutation, a missense mutation, or a nonsense mutation in the endogenous genes.

In a particular embodiment the cultivated crop plant has an increased yield.

In a particular embodiment the invention provides a cultivated crop plant as herein defined before with the proviso that the cultivated crop plant is not exclusively produced by an essentially biological method.

In another particular embodiment the invention provides a cultivated crop plant as herein described before wherein the cultivated crop plant is selected from the group consisting of rice, wheat, barley, corn, soybean, cotton, sugarcane, sorghum, millet, rye, oats, cocoa, beans, grape, tomato, cassava, castor bean, poplar, eucalyptus, papaya, and oilseed.

These plants and parts can be utilized for human food, livestock feed, as a raw material in industry, or as breeding material for development of cultivated crop plants.

In a particular embodiment the invention provides a corn plant which has a combined loss-of-function allele in GRF4, which leads to a loss-of-expression of the GRF4 protein (SEQ ID NO: 7) and a loss-of-function in TCP9, which leads to a loss-of-expression of the TCP9 protein (SEQ ID NO: 21).

In yet another embodiment the invention provides a corn plant which has a combined loss-of-function allele in GRF10, which leads to a loss-of-expression of the GRF10 protein (SEQ ID NO: 8) and a loss-of-function in TCP42, which leads to a loss-of-expression of the TCP42 protein (SEQ ID NO: 30).

GRF4 and GRF10 are two genes from corn which encode for two orthologous proteins (SEQ ID NO: 7 and SEQ ID NO: 8). The orthologous plant proteins, GRF4 and GRF10, have two consensus sequences depicted in SEQ ID NO: 1 and 2 which are present in monocot and dicot plants. Orthologous amino acid sequences of GRF4 and GRF10 from plants are depicted in SEQ ID NO: 9 to 20.

TCP42 is a gene from corn which encodes for a protein depicted in SEQ ID NO: 30. Orthologous plant proteins of TCP42 have two consensus sequences depicted in SEQ ID NO: 3 and 4 which are presented in monocot and dicot plants. Orthologous amino acid sequences of TCP42 from plants are depicted in SEQ ID NO: 31-45.

TCP9 is a gene from corn which encodes for a protein depicted in SEQ ID NO: 21. Orthologous plant proteins of TCP9 have two consensus sequences depicted in SEQ ID NO: 5 and 6 which are presented in monocot and dicot plants. Orthologous amino acid sequences of TCP9 from plants are depicted in SEQ ID NO: 22-29.

GRF10/GRF4 Orthologous Amino Acid Sequences from Plants:

Multilevel PEPGRCRRTDGKKWRCSRDAIPNEKYCERHMHRGRKR
consensus WKNTV DQ N H
sequence K
SEQ ID NO: 1: PEPGRCRRTDGKKWRCX₁X₂X₃X₄X₅PX₆X₇KYCERHMX₈RGRX₉R

Wherein X1 is S or W, X2 is R or K, X3 is D or N, X4 is A, Tor K, X5 is I or V, X6 is N or D, X7 is E or Q, X8 is H or N and X9 is K or H

Multilevel AAFTAMQLQELEQQSLIYKYMAARVPVPTHL
consensus GP W QL ARV Q IT N
sequence CS G
SEQ ID NO: 2: X₁X₂FTAMQX₃QELX₄X₅QX₆X₇X₈YX₉YX₁₀X₁₁AX₁₂VPVPTHL

Wherein X1 is A, G or C, X2 is A, P or S, X3 is L or W, X4 is E or Q, X5 is Q or L, X6 is S or A, X7 is L or R, X8 is I or V, X9 is K or Q, X10 is M or I, X11 is A or T and X12 is R, N or G

TCP42 Orthologous Amino Acid Sequences from Plants:

Multilevel GGKDRHSKVVTSRGLRDRRVRLSVPTAIQFYDLQDRLG
consensus M AK I L I
sequence
SEQ ID NO: 3: GGKDRHSKVX₁TX₂X₃GLRDRRX₄RLSVPTAIQX₅YDX₆QDRLG

Wherein X1 is V or M, X2 is S or A, X3 is R or K, X4 is V or I, X5 is F or L and X6 is L or I

Multilevel ANQSQNPSAAKSACSSTSETSKGSVLSL
consensus PVASLALED GEV G
sequence S
SEQ ID NO: 4: ANX₁X₂X₃X₄X₅X₆X₇X₈X₉SX₁₀X₁₁X₁₂STSETSKGSX₁₃LSL

Wherein X1 is Q or P, X2 is S or V, X3 is Q or A, X4 is N or S, X5 is P or L, X6 is S or A, X7 is A or L, X8 is A, E or S, X9 is K or D, X10 is A or G, X11 is C or E, X12 is S or V and X13 is V or G.

Orthologous TCP9 Amino Acid Sequences from Plants:

Multilevel RKDRHSKVCTARGPRDRRVRLSAHTAIQFYDVQDRLGYDRPSKAVDWLIK
consensus K A
sequence
SEQ ID NO: 5: RKDRHSKVCTAX₁GPRDRRVRLX₂AHTAIQFYDVQDRLGYDRPSKAVDWLIK

Wherein X1 is R or K, X2 is S or A

Multilevel EASSSTVAAHSSAMGFQGYTPDLLSRTGSQSQELRLSLQSLPDPMFHHH
consensus P A TQ S NH P Q
sequence T
SEQ ID NO: 6: EX₁SSSTX₂AX₃X₄SSAMGFQX₅YTPDLLSRTGX₆X₇SQELRLSLQX₈LPDPMFHH

Wherein X1 is A or P, X2 is V, A or T, X3 is A or T, X4 is H or Q, X5 is G or S, X6 is S or N, X7 is Q or H and X8 is

In certain embodiments, the methods can comprise making a deletion, an insertion and/or a substitution which results in a loss-of-function allele of the endogenous crop gene. Gene editing molecules of use in methods provided herein include molecules capable of introducing a double-strand break (“DSB”) or single-strand break (“SSB”) at a specific site or sequence in a double-stranded DNA, such as in genomic DNA or in a target gene located within the genomic DS or PNA as well as accompanying guide RNA. In certain embodiments, the loss-of-function allele results from introduction of a DSB at a target site in the endogenous crop gene to induce non-homologous end joining (NHEJ) at the site of the break followed by recovery of desired loss-of-function alleles. In certain embodiments, the loss-of-function allele results from introduction of a DSB at a target site in the endogenous crop gene followed by homology-directed repair (HDR), microhomology-mediated end joining (MMEJ), or NHEJ to introduce a desired donor or other DNA template polynucleotide at the DSB, followed by recovery of the desired loss-of-function allele. Examples of such gene editing molecules include: (a) a nuclease comprising an RNA-guided nuclease, an RNA-guided DNA endonuclease or RNA directed DNA endonuclease (RdDe), a class 1 CRISPR type nuclease system, a type II Cas nuclease, a Cas9, a nCas9 nickase, a type V Cas nuclease, a Cas12a nuclease, a nCas12a nickase, a Cas12d (CasY), a Cas12e (CasX), a Cas12b (C2c1), a Cas12c (C2c3), a Cas12i, a Cas12j, a Cas14, an engineered nuclease, a codon-optimized nuclease, a zinc-finger nuclease (ZFN) or nickase, a transcription activator-like effector nuclease (TAL-effector nuclease or TALEN) or nickase (TALE-nickase), an Argonaute, and a meganuclease or engineered meganuclease; (b) a polynucleotide encoding one or more nucleases capable of effectuating site-specific alteration (including introduction of a DSB or SSB) of a target nucleotide sequence; (c) a guide RNA (gRNA) for use with an RNA-guided nuclease, or a DNA encoding a gRNA for use with an RNA-guided nuclease; (d) optionally donor DNA template polynucleotides suitable for insertion at a break in genomic DNA by homology-directed repair (HDR) or microhomology-mediated end joining (MMEJ); and (e) optionally other DNA templates (e.g., dsDNA, ssDNA, or combinations thereof) suitable for insertion at a break in genomic DNA (e.g., by non-homologous end joining (NHEJ). In certain embodiments, the loss-of-function allele of the endogenous crop gene and plant cells, parts including seeds, and plants comprising the loss-of-function allele of the endogenous crop gene are generated by CRISPR technology. CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1. Plant RNA promoters for expressing CRISPR guide RNA and plant codon-optimized CRISPR Cas9 endonuclease are disclosed in published WO 2015/131101. Methods of using CRISPR technology for genome editing in plants are disclosed in US Patent Application Publications US 2015/0082478A1 and US 2015/0059010A1 and in published WO 2016/007347. In certain embodiments, an RNA-guided endonuclease that leaves a blunt end following cleavage of the target site is used. Blunt-end cutting RNA-guided endonucleases include Cas9, Cas12c, Cas12i, and Cas 12 h (Yan et al., 2019). In certain embodiments, an RNA-guided endonuclease that leaves a staggered single stranded DNA overhanging end following cleavage of the target site following cleavage of the target site is used. Staggered-end cutting RNA-guided endonucleases include Cas12a, Cas12b, and Cas12e. Guide RNA molecules comprising a spacer RNA molecule which targets the endogenous crop genes or an allelic variant thereof are provided. CRISPR-type genome editing can be adapted for use in the plant cells and methods provided herein in several ways. CRISPR elements, e.g., gene editing molecules comprising CRISPR endonucleases and CRISPR guide RNAs including single guide RNAs or guide RNAs in combination with tracrRNAs or scoutRNA, or polynucleotides encoding the same, are useful in effectuating genome editing without remnants of the CRISPR elements or selective genetic markers occurring in progeny. In certain embodiments, the CRISPR elements are provided directly to the eukaryotic cell (e.g., maize plant cells), systems, methods, and compositions as isolated molecules, as isolated or semi-purified products of a cell free synthetic process (e.g., in vitro translation), or as isolated or semi-purified products of in a cell-based synthetic process (e.g., such as in a bacterial or other cell lysate). In certain embodiments, crop plants or crop plant cells used in the systems, methods, and compositions provided herein can comprise a transgene that expresses a CRISPR endonuclease (e.g., a Cas9, a Cpf1-type or other CRISPR endonuclease). In certain embodiments, one or more CRISPR endonucleases with unique PAM recognition sites can be used. Guide RNAs (sgRNAs or crRNAs and a tracrRNA) to form an RNA-guided endonuclease/guide RNA complex which can specifically bind sequences in the gDNA target site that are adjacent to a protospacer adjacent motif (PAM) sequence. The type of RNA-guided endonuclease typically informs the location of suitable PAM sites and design of crRNAs or sgRNAs. G-rich PAM sites, e.g., 5′-NGG are typically targeted for design of crRNAs or sgRNAs used with Cas9 proteins. Examples of PAM sequences include 5′-NGG (Streptococcus pyogenes), 5′-NNAGAA (Streptococcus thermophilus CRISPR1), 5′-NGGNG (Streptococcus thermophilus CRISPR3), 5′-NNGRRT or 5′-NNGRR (Staphylococcus aureus Cas9, SaCas9), and 5′-NNNGATT (Neisseria meningitidis). T-rich PAM sites (e.g., 5′-TTN or 5′-TTTV, where “V” is A, C, or G) are typically targeted for design of crRNAs or sgRNAs used with Cas12a proteins. In some instances, Cas12a can also recognize a 5′-CTA PAM motif. Other examples of potential Cas12a PAM sequences include TTN, CTN, TCN, CCN, TITN, TCTN, TTCN, CTTN, ATTN, TCCN, TTGN, GTTN, CCCN, CCTN, TTAN, TCGN, CTCN, ACTN, GCTN, TCAN, GCCN, and CCGN (wherein N is defined as any nucleotide). Cpf1 endonuclease and corresponding guide RNAs and PAM sites are disclosed in US Patent Application Publication 2016/0208243 A1, which is incorporated herein by reference for its disclosure of DNA encoding Cpf1 endonucleases and guide RNAs and PAM sites.

In certain embodiments, the loss-of-function allele of the endogenous crop gene and plant cells, parts including seeds, and plants comprising the loss-of-function allele of the endogenous crop gene are generated by use of zinc finger nucleases or zinc finger nickases. Zinc-finger nucleases are site-specific endonucleases comprising two protein domains: a DNA-binding domain, comprising a plurality of individual zinc finger repeats that each recognize between 9 and 18 base pairs, and a DNA-cleavage domain that comprises a nuclease domain (typically Fokl). The cleavage domain dimerizes in order to cleave DNA; therefore, a pair of ZFNs are required to target non-palindromic target polynucleotides. In certain embodiments, zinc finger nuclease and zinc finger nickase design methods which have been described (Urnov et al. (2010) Nature Rev. Genet., 11:636-646; Mohanta et al. (2017) Genes vol. 8,12:399; Ramirez et al. Nucleic Acids Res. (2012); 40 (12): 5560-5568; Liu et al. (2013) Nature Communications, 4:2565) can be adapted for use in the methods set forth herein. The zinc finger binding domains of the zinc finger nuclease or nickase provide specificity and can be engineered to specifically recognize any desired target DNA sequence. The zinc finger DNA binding domains are derived from the DNA-binding domain of a large class of eukaryotic transcription factors called zinc finger proteins (ZFPs). The DNA-binding domain of ZFPs typically contains a tandem array of at least three zinc “fingers” each recognizing a specific triplet of DNA. A number of strategies can be used to design the binding specificity of the zinc finger binding domain. One approach, termed “modular assembly”, relies on the functional autonomy of individual zinc fingers with DNA. In this approach, a given sequence is targeted by identifying zinc fingers for each component triplet in the sequence and linking them into a multifinger peptide. Several alternative strategies for designing zinc finger DNA binding domains have also been developed. These methods are designed to accommodate the ability of zinc fingers to contact neighboring fingers as well as nucleotide bases outside their target triplet. Typically, the engineered zinc finger DNA binding domain has a novel binding specificity, compared to a naturally occurring zinc finger protein. Engineering methods include, for example, rational design and various types of selection. Rational design includes, for example, the use of databases of triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, e.g., U.S. Pat. Nos. 6,453,242 and 6,534,261, both incorporated herein by reference in their entirety. Exemplary selection methods (e.g., phage display and yeast two-hybrid systems) can be adapted for use in the methods described herein. In addition, enhancement of binding specificity for zinc finger binding domains has been described in U.S. Pat. No. 6,794,136, incorporated herein by reference in its entirety. In addition, individual zinc finger domains may be linked together using any suitable linker sequences. Examples of linker sequences are publicly known, e.g., see U.S. Pat. Nos. 6,479,626; 6,903,185; and 7,153,949, incorporated herein by reference in their entirety. The nucleic acid cleavage domain is non-specific and is typically a restriction endonuclease, such as Fokl. This endonuclease must dimerize to cleave DNA. Thus, cleavage by Fokl as part of a ZFN requires two adjacent and independent binding events, which must occur in both the correct orientation and with appropriate spacing to permit dimer formation. The requirement for two DNA binding events enables more specific targeting of long and potentially unique recognition sites. Fokl variants with enhanced activities have been described and can be adapted for use in the methods described herein; see, e.g., Guo et al. (2010) J. Mol. Biol., 400:96-107.

In certain embodiments, the loss-of-function allele of the endogenous crop gene and plant cells, parts including seeds, and plants comprising the loss-of-function allele of the endogenous crop gene are generated by use of TAL-effector nucleases or TALENs. Transcription activator like effectors (TALEs) are proteins secreted by certain Xanthomonas species to modulate gene expression in host plants and to facilitate the colonization by and survival of the bacterium. TALEs act as transcription factors and modulate expression of resistance genes in the plants. Recent studies of TALEs have revealed the code linking the repetitive region of TALEs with their target DNA-binding sites. TALEs comprise a highly conserved and repetitive region consisting of tandem repeats of mostly 33 or 34 amino acid segments. The repeat monomers differ from each other mainly at amino acid positions 12 and 13. A strong correlation between unique pairs of amino acids at positions 12 and 13 and the corresponding nucleotide in the TALE-binding site has been found. The simple relationship between amino acid sequence and DNA recognition of the TALE binding domain allows for the design of DNA binding domains of any desired specificity. TALEs can be linked to a non-specific DNA cleavage domain to prepare genome editing proteins, referred to as TAL-effector nucleases or TALENs. As in the case of ZFNs, a restriction endonuclease, such as Fokl, can be conveniently used. Methods for use of TALENs in plants have been described and can be adapted for use in the methods described herein, see Mahfouz et al. (2011) Proc. Natl. Acad. Sci. USA, 108:2623-2628; Mahfouz (2011) GM Crops, 2:99-103; and Mohanta et al. (2017) Genes vol. 8,12:399). TALE nickases have also been described and can be adapted for use in methods described herein (Wu et al.; Biochem Biophys Res Commun. (2014); 446 (1): 261-6; Luo et al; Scientific Reports 6, Article number: 20657 (2016)). Various treatments can be used for delivery of gene editing molecules and/or other molecules to a plant cell. In certain embodiments, one or more treatments is employed to deliver the gene editing or other molecules (e.g., comprising a polynucleotide, polypeptide or combination thereof) into a plant cell, e.g., through barriers such as a cell wall, a plasma membrane, a nuclear envelope, and/or other lipid bilayer. In certain embodiments, a polynucleotide-, polypeptide-, or RNP (ribonucleoprotein)-containing composition comprising the molecules are delivered directly, for example by direct contact of the composition with a plant cell. Aforementioned compositions can be provided in the form of a liquid, a solution, a suspension, an emulsion, a reverse emulsion, a colloid, a dispersion, a gel, liposomes, micelles, an injectable material, an aerosol, a solid, a powder, a particulate, a nanoparticle, or a combination thereof can be applied directly to a plant, plant part, plant cell, or plant explant (e.g., through abrasion or puncture or otherwise disruption of the cell wall or cell membrane, by spraying or dipping or soaking or otherwise directly contacting, by microinjection). For example, a plant cell or plant protoplast is soaked in a liquid genome editing molecule-containing composition. In certain embodiments, the composition is delivered using negative or positive pressure, for example, using vacuum infiltration or application of hydrodynamic or fluid pressure. In certain embodiments, the composition is introduced into a plant cell or plant protoplast, e.g., by microinjection or by disruption or deformation of the cell wall or cell membrane, for example by physical treatments such as by application of negative or positive pressure, shear forces, or treatment with a chemical or physical delivery agent such as surfactants, liposomes, or nanoparticles; see, e.g., delivery of materials to cells employing microfluidic flow through a cell-deforming constriction as described in US Published Patent Application 2014/0287509, incorporated by reference in its entirety herein. Other techniques useful for delivering the composition to a eukaryotic cell, plant cell or plant protoplast include: ultrasound or sonication; vibration, friction, shear stress, vortexing, cavitation; centrifugation or application of mechanical force; mechanical cell wall or cell membrane deformation or breakage; enzymatic cell wall or cell membrane breakage or permeabilization; abrasion or mechanical scarification (e.g., abrasion with carborundum or other particulate abrasive or scarification with a file or sandpaper) or chemical scarification (e.g., treatment with an acid or caustic agent); and electroporation. In certain embodiments, the composition is provided by bacterially mediated (e.g., Agrobacterium sp., Rhizobium sp., Sinorhizobium sp., Mesorhizobium sp., Bradyrhizobium sp., Azobacter sp., Phyllobacterium sp.) transfection of the plant cell or plant protoplast with a polynucleotide encoding the genome editing molecules (e.g., RNA dependent DNA endonuclease, RNA dependent DNA binding protein, RNA dependent nickase, ABE, or CBE, and/or guide RNA); see, e.g., Broothaerts et al. (2005) Nature, 433:629-633). Any of these techniques or a combination thereof are alternatively employed on a plant explant, plant part or tissue or intact plant (or seed) from which a plant cell is optionally subsequently obtained or isolated; in certain embodiments, the composition is delivered in a separate step after the plant cell has been isolated.

In certain embodiments, the population of crop plant cells, parts, or plants which are screened for the presence of a loss-of-function allele in the endogenous crop gene have been subjected to one or more mutagenesis treatments. Loss-of-function alleles of the endogenous crop gene can be generated by mutagenesis methods known in the art, such as chemical mutagenesis or radiation mutagenesis. Suitable chemical mutagens include ethyl methanesulfonate (EMS), sodium azide, methylnitrosourea (MNU), and diepoxybutane (DEB). Suitable radiation includes x-rays, fast neutron radiation, and gamma radiation. Crop plant cells, parts, or plants comprising a loss-of-function allele of the endogenous crop gene can be generated using mutagenesis and identified by TILLING (Targeting Induced Local Lesions IN Genomes) or identified using EcoTILLING. TILLING is a general reverse genetics technique that uses mutagenesis methods to create libraries of mutagenized individuals that are later subjected to high throughput screens for the discovery of mutations. In addition to allowing efficient detection of induced mutations, high-throughput TILLING technology is ideal for the detection of natural mutations. EcoTILLING is a method that uses TILLING techniques to look for natural mutations in individuals (Barkley and Wang. Current genomics vol. 9,4 (2008): 212-26. doi: 10.2174/138920208784533656). Identified mutations can then be introduced into desirable genetic backgrounds by crossing the mutant with a plant of the desired genetic background and performing a suitable number of backcrosses to cross out the originally undesired parent background. A more detailed description of methods and compositions for TILLING are disclosed in US Patent Application Publication 2004/0053236 A1, which is incorporated herein by reference in its entirety and can be adapted for use in the methods provided herein for identifying crop plant cells, parts, or plants comprising a loss-of-function allele of the endogenous crop gene. This disclosure is also directed to methods for producing a crop plant having a loss-of-function allele of the endogenous crop gene by crossing a first parent crop plant with a second parent crop plant wherein the first or second parent crop plant comprises the loss-of-function allele. Further, both the first and second parent crop plants can comprise the loss-of-function allele. Any such methods using a crop plant comprising the loss-of-function allele are part of this disclosure: selfing, backcrosses, hybrid production, crosses to populations, and the like. All plants produced using a crop plant comprising the loss-of-function allele as a parent are within the scope of this disclosure, including plants derived from a crop plant having the loss-of-function allele. Also provided are the F1 progeny crop plants produced from the crossing of a crop plant comprising the loss-of-function allele with any other crop plant, F1 seed, and various parts of the F1 crop plant. The following describes breeding methods that can be used with crop plants of the disclosure in the development of further crop plants. One such embodiment is a method for developing a progeny crop plant in a crop plant breeding program comprising: obtaining the crop plant, or its parts, comprising a loss-of-function allele of the endogenous crop gene and utilizing the plant or plant parts as a source of breeding material; and selecting a progeny plant having the loss-of-function allele. Breeding steps that can be used in the crop plant breeding program include pedigree breeding, backcrossing, mutation breeding, and recurrent selection. In conjunction with these steps, techniques such as restriction fragment polymorphism enhanced selection, marker-assisted selection (for example SNP or SSR markers), and the making of double haploids can be utilized.

Field crops are bred through techniques that take advantage of the plant's method of pollination. A crop plant of the disclosure can be self-pollinated, sib-pollinated, or cross pollinated to create a pedigree crop plant. A plant is self-pollinated if pollen from one flower is transferred to the same or another flower of the same plant. A plant is sib-pollinated when individuals within the same family or variety are used for pollination. A plant is cross-pollinated if the pollen comes from a flower on a different plant from a different family or variety. The terms “cross-pollination” and “out-cross” as used herein do not include self-pollination or sib-pollination. For example maize can be bred by both self-pollination and cross-pollination techniques. Maize has separate male and female flowers on the same plant, located on the tassel and the ear, respectively. Natural pollination occurs in maize when wind blows pollen from the tassels to the silks that protrude from the tops of the ears.

Any other suitable breeding, selection, or growing methods may be used. Choice of the particular breeding or selection method will vary depending on environmental factors, population size, and the like.

In certain optional embodiments, the crop plant cells disclosed herein are non-regenerable crop plant cells. In certain optional embodiments provided herein, the crop plant cells, plant propagules (e.g., a seed, seedling, ovule, embryo, pollen, root, stem, leaf, shoot, explant, or callus), and plants provided herein are not produced by an exclusively biological process. In certain optional embodiments provided herein, the methods for producing crop plant cells, plant propagules (e.g., a seed, seedling, ovule, embryo, pollen, root, stem, leaf, shoot, explant, or callus), and plants provided herein are not exclusively biological processes.

The term “yield” as used herein generally refers to a measurable product from a plant, particularly a crop. Yield and yield increase (in comparison to a non-transformed starting or wild-type plant) can be measured in a number of ways, and it is understood that a skilled person will be able to apply the correct meaning in view of the particular embodiments, the particular crop concerned and the specific purpose or application concerned. The terms “improved yield” or “increased yield” can be used interchangeable. As used herein, the term “improved yield” or the term “increased yield” means any improvement in the yield of any measured plant product, such as grain, fruit, leaf, root, cob or fiber. In accordance with the invention, changes in different phenotypic traits may improve yield. For example, and without limitation, parameters such as floral organ development, root initiation, root biomass, seed number, seed weight, harvest index, leaf formation, phototropism, apical dominance, and fruit development, are suitable measurements of improved yield. Increased yield includes higher fruit yields, higher seed yields, higher fresh matter production, and/or higher dry matter production. Any increase in yield is an improved yield in accordance with the invention. For example, the improvement in yield can comprise a 0.1%, 0.5%, 1%, 3%, 5%, 10%, 15%, 20%, 30%, 40%, 50%, 60%, 70%, 80%, 90% or greater increase in any measured parameter. For example, an increase in the bu/acre yield of soybeans or corn derived from a crop comprising plants which are transgenic for the chimeric genes of the invention, as compared with the bu/acre yield from untransformed soybeans or corn cultivated under the same conditions, is an improved yield in accordance with the invention. The increased or improved yield can be achieved in the absence or presence of stress conditions. For example, enhanced or increased “yield” refers to one or more yield parameters selected from the group consisting of biomass yield, dry biomass yield, aerial dry biomass yield, underground dry biomass yield, fresh-weight biomass yield, aerial fresh-weight biomass yield, underground fresh-weight biomass yield; enhanced yield of harvestable parts, either dry or fresh-weight or both, either aerial or underground or both; enhanced yield of crop fruit, either dry or fresh-weight or both, either aerial or underground or both; and enhanced yield of seeds, either dry or fresh-weight or both, either aerial or underground or both. “Crop yield” is defined herein as the number of bushels of relevant agricultural product (such as grain, forage, or seed) harvested per acre. Crop yield is impacted by abiotic stresses, such as drought, heat, salinity, and cold stress, and by the size (biomass) of the plant. The yield of a plant can depend on the specific plant/crop of interest as well as its intended application (such as food production, feed production, processed food production, biofuel, biogas or alcohol production, or the like) of interest in each particular case. Thus, in one embodiment, yield can be calculated as harvest index (expressed as a ratio of the weight of the respective harvestable parts divided by the total biomass), harvestable parts weight per area (acre, square meter, or the like); and the like. The harvest index is the ratio of yield biomass to the total cumulative biomass at harvest. Harvest index is relatively stable under many environmental conditions, and so a robust correlation between plant size and grain yield is possible. Measurements of plant size in early development, under standardized conditions in a growth chamber or greenhouse, are standard practices to measure potential yield advantages conferred by the presence of a transgene. Accordingly, the yield of a plant can be increased by improving one or more of the yield-related phenotypes or traits. Such yield-related phenotypes or traits of a plant the improvement of which results in increased yield comprise, without limitation, the increase of the intrinsic yield capacity of a plant, improved nutrient use efficiency, and/or increased stress tolerance. For example, yield refers to biomass yield, e.g. to dry weight biomass yield and/or fresh-weight biomass yield. Biomass yield refers to the aerial or underground parts of a plant, depending on the specific circumstances (test conditions, specific crop of interest, application of interest, and the like). In one embodiment, biomass yield refers to the aerial and underground parts. Biomass yield may be calculated as fresh-weight, dry weight or a moisture adjusted basis. Biomass yield may be calculated on a per plant basis or in relation to a specific area (e.g. biomass yield per acre/square meter/or the like). “Yield” can also refer to seed yield which can be measured by one or more of the following parameters: number of seeds or number of filled seeds (per plant or per area (acre/square meter/or the like)); seed filling rate (ratio between number of filled seeds and total number of seeds); number of flowers per plant; seed biomass or total seeds weight (per plant or per area (acre/square meter/or the like); thousand kernel weight (TKW; extrapolated from the number of filled seeds counted and their total weight; an increase in TKW may be caused by an increased seed size, an increased seed weight, an increased embryo size, and/or an increased endosperm). Other parameters allowing to measure seed yield are also known in the art. Seed yield may be determined on a dry weight or on a fresh weight basis, or typically on a moisture adjusted basis, e.g. at 15.5 percent moisture. For example, the term “increased yield” means that a plant, exhibits an increased growth rate, e.g. in the absence or presence of abiotic environmental stress, compared to the corresponding wild-type plant. An increased growth rate may be reflected inter alia by or confers an increased biomass production of the whole plant, or an increased biomass production of the aerial parts of a plant, or by an increased biomass production of the underground parts of a plant, or by an increased biomass production of parts of a plant, like stems, leaves, blossoms, fruits, and/or seeds. A prolonged growth comprises survival and/or continued growth of the plant, at the moment when the non-transformed wild type organism shows visual symptoms of deficiency and/or death. When the plant of the invention is a corn plant, increased yield for corn plants means, for example, increased seed yield, in particular for corn varieties used for feed or food. Increased seed yield of corn refers to an increased kernel size or weight, an increased kernel per ear, or increased ears per plant. Alternatively or in addition the cob yield may be increased, or the length or size of the cob is increased, or the kernel per cob ratio is improved.

When the plant of the invention is a soy plant, increased yield for soy plants means increased seed yield, in particular for soy varieties used for feed or food. Increased seed yield of soy refers for example to an increased kernel size or weight, an increased kernel per pod, or increased pods per plant. When the plant of the invention is an oil seed rape (OSR) plant, increased yield for OSR plants means increased seed yield, in particular for OSR varieties used for feed or food. Increased seed yield of OSR refers to an increased seed size or weight, an increased seed number per silique, or increased siliques per plant. When the plant of the invention is a cotton plant, increased yield for cotton plants means increased lint yield. Increased lint yield of cotton refers in one embodiment to an increased length of lint. When the plant is a plant belonging to grasses an increased leaf can mean an increased leaf biomass. Said increased yield can typically be achieved by enhancing or improving, one or more yield-related traits of the plant. Such yield-related traits of a plant comprise, without limitation, the increase of the intrinsic yield capacity of a plant, improved nutrient use efficiency, and/or increased stress tolerance, in particular increased abiotic stress tolerance. Intrinsic yield capacity of a plant can be, for example, manifested by improving the specific (intrinsic) seed yield (e.g. in terms of increased seed/grain size, increased ear number, increased seed number per ear, improvement of seed filling, improvement of seed composition, embryo and/or endosperm improvements, or the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under stressed or non-stressed conditions), improvement in plant architecture, cell cycle modifications, photosynthesis modifications, various signaling pathway modifications, modification of transcriptional regulation, modification of translational regulation, modification of enzyme activities, and the like); and/or the like

“Selectable marker”, “selectable marker gene” or “reporter gene” includes any gene that confers a phenotype on a cell in which it is expressed to facilitate the identification and/or selection of cells that are transfected or transformed with a nucleic acid construct of the invention. These marker genes enable the identification of a successful transfer of the nucleic acid molecules via a series of different principles. Suitable markers may be selected from markers that confer antibiotic or herbicide resistance, that introduce a new metabolic trait or that allow visual selection. Examples of selectable marker genes include genes conferring resistance to antibiotics (such as nptll that phosphorylates neomycin and kanamycin, or hpt, phosphorylating hygromycin, or genes conferring resistance to, for example, bleomycin, streptomycin, tetracyclin, chloramphenicol, ampicillin, gentamycin, geneticin (G418), spectinomycin or blasticidin), to herbicides (for example bar which provides resistance to Basta*; aroA or gox providing resistance against glyphosate, or the genes conferring resistance to, for example, imidazolinone, phosphinothricin or sulfonylurea), or genes that provide a metabolic trait (such as manA that allows plants to use mannose as sole carbon source or xylose isomerase for the utilisation of xylose, or antinutritive markers such as the resistance to 2-deoxyglucose). Expression of visual marker genes results in the formation of colour (for example B-glucuronidase, GUS or B-galactosidase with its coloured substrates, for example X-Gal), luminescence (such as the luciferin/luciferase system) or fluorescence (Green Fluorescent Protein, GFP, and derivatives thereof). This list represents only a small number of possible markers. The skilled worker is familiar with such markers. Different markers are preferred, depending on the organism and the selection method.

It is known that upon stable or transient integration of nucleic acids into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can for example be used in mutants in which these genes are not functional by, for example, deletion by conventional methods. Furthermore, nucleic acid molecules encoding a selectable marker can be introduced into a host cell on the same vector that comprises the sequence encoding the polypeptides of the invention or used in the methods of the invention, or else in a separate vector. Cells which have been stably transfected with the introduced nucleic acid can be identified for example by selection (for example, cells which have integrated the selectable marker survive whereas the other cells die).

Since the marker genes, particularly genes for resistance to antibiotics and herbicides, are no longer required or are undesired in the transgenic host cell once the nucleic acids have been introduced successfully, the process according to the invention for introducing the nucleic acids advantageously employs techniques which enable the removal or excision of these marker genes. One such a method is what is known as co-transformation. The co-transformation method employs two vectors simultaneously for the transformation, one vector bearing the nucleic acid according to the invention and a second bearing the marker gene(s). A large proportion of transformants receives or, in the case of plants, comprises (up to 40% or more of the transformants), both vectors. In case of transformation with Agrobacteria, the transformants usually receive only a part of the vector, i.e. the sequence flanked by the T-DNA, which usually represents the expression cassette. The marker genes can subsequently be removed from the transformed plant by performing crosses. In another method, marker genes integrated into a transposon are used for the transformation together with desired nucleic acid (known as the Ac/Ds technology). The transformants can be crossed with a transposase source or the transformants are transformed with a nucleic acid construct conferring expression of a transposase, transiently or stable. In some cases (approx. 10%), the transposon jumps out of the genome of the host cell once transformation has taken place successfully and is lost. In a further number of cases, the transposon jumps to a different location. In these cases the marker gene must be eliminated by performing crosses. In microbiology, techniques were developed which make possible, or facilitate, the detection of such events. A further advantageous method relies on what is known as recombination systems; whose advantage is that elimination by crossing can be dispensed with. The best-known system of this type is what is known as the Cre/lox system. Cre1 is a recombinase that removes the sequences located between the loxP sequences. If the marker gene is integrated between the loxP sequences, it is removed once transformation has taken place successfully, by expression of the recombinase. Further recombination systems are the HIN/HIX, FLP/FRT and REP/STB system (Tribble et al., J. Biol. Chem., 275, 2000:22255-22267; Velmurugan et al., J. Cell Biol., 149, 2000:553-566). A site-specific integration into the plant genome of the nucleic acid sequences according to the invention is possible.

The term “expression” or “gene expression” means the transcription of a specific gene or specific genes or specific genetic construct. The term “expression” or “gene expression” in particular means the transcription of a gene or genes or genetic construct into structural RNA (rRNA, tRNA) or mRNA with or without subsequent translation of the latter into a protein. The process includes transcription of DNA and processing of the resulting mRNA product.

The term “introduction” or “transformation” as referred to herein encompass the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Biol 8:363-373); electroporation of protoplasts (Shillito R. D. et al. (1985) Bio/Technol 3, 1099-1 102); microinjection into plant material (Crossway A et al., (1986) Mol. Gen Genet 202:179-185); DNA or RNA-coated particle bombardment (Klein T M et al., (1987) Nature 327:70) infection with (non-integrative) viruses and the like. Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least on the flower primordia. The plant is subsequently grown on until the seeds of the treated plant are obtained (Clough and Bent, Plant J. (1998) 16, 735-743). Methods for Agrobacterium-mediated transformation of rice include well known methods for rice transformation, such as those described in any of the following: European patent application EP1198985, Aldemita and Hodges (Planta 199:612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14 (6): 745-50, 1996) or Frame et al. (Plant Physiol 129 (1): 13-22, 2002), which disclosures are incorporated by reference herein as if fully set forth. Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acids or the construct to be expressed is preferably cloned into a vector, which is suitable for transforming Agrobacterium tumefaciens, for example pBin19 (Bevan et al (1984) Nucl. Acids Res. 12-8711). Agrobacteria transformed by such a vector can then be used in known manner for the transformation of plants, such as plants used as a model, like Arabidopsis (Arabidopsis thaliana is within the scope of the present invention not considered as a crop plant), or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Hofgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

In addition to the transformation of somatic cells, which then have to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic [Feldman, KA and Marks MD (1987). Mol Gen Genet 208:1-9; Feldmann K (1992). In: C Koncz, N—H Chua and J Shell, eds, Methods in Arabidopsis Research. Word Scientific, Singapore, pp. 274-289]. Alternative methods are based on the repeated removal of the inflorescences and incubation of the excision site in the center of the rosette with transformed agrobacteria, whereby transformed seeds can likewise be obtained at a later point in time (Chang (1994). Plant J. 5:551-558; Katavic (1994). Mol Gen Genet, 245:363-370). However, an especially effective method is the vacuum infiltration method with its modifications such as the “floral dip” method. In the case of vacuum infiltration of Arabidopsis, intact plants under reduced pressure are treated with an agrobacterial suspension [Bechthold, N (1993). CR

Acad Sci Paris Life Sci, 316:1 194-1 199], while in the case of the “floral dip” method the developing floral tissue is incubated briefly with a surfactant-treated agrobacterial suspension [Clough, S J and Bent A F (1998) The Plant J. 16, 735-743]. A certain proportion of transgenic seeds are harvested in both cases, and these seeds can be distinguished from non-transgenic seeds by growing under the above-described selective conditions. In addition the stable transformation of plastids is of advantages because plastids are inherited maternally is most crops reducing or eliminating the risk of transgene flow through pollen. The transformation of the chloroplast genome is generally achieved by a process which has been schematically displayed in Klaus et al., 2004 [Nature Biotechnology 22 (2), 225-229]. Briefly the sequences to be transformed are cloned together with a selectable marker gene between flanking sequences homologous to the chloroplast genome. These homologous flanking sequences direct site specific integration into the plastome. Plastidal transformation has been described for many different plant species and an overview is given in Bock (2001) Transgenic plastids in basic research and plant biotechnology. J Mol Biol. 2001 Sep. 21; 312 (3): 425-38 or Maliga, P (2003) Progress towards commercialization of plastid transformation technology. Trends Biotechnol. 21, 20-28. Further biotechnological progress has recently been reported in form of marker free plastid transformants, which can be produced by a transient co-integrated maker gene (Klaus et al., 2004, Nature Biotechnology 22 (2), 225-229).

The genetically modified plant cells can be regenerated via all methods with which the skilled worker is familiar. Suitable methods can be found in the abovementioned publications by S. D. Kung and R. Wu, Potrykus or Hofgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The terms “increase”, “improve” or “enhance” are interchangeable and shall mean in the sense of the application at least a 3%, 4%, 5%, 6%, 7%, 8%, 9% or 10%, preferably at least 15% or 20%, more preferably 25%, 30%, 35%, 40%, 50%, 60%, 70%, 80%, 90%, 95% or more yield and/or growth in comparison to control plants as defined herein.

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

Plants that are particularly useful in the methods of the invention include in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

### Examples

### 1. Efficient Maize Haploid Induction and Embryo Rescue Doubling Using the B104 Inbred Line

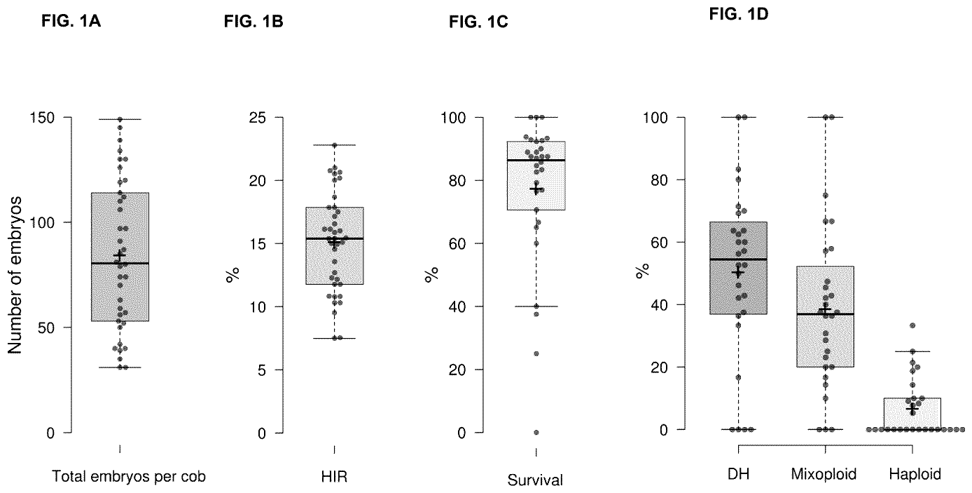

We used the HI line RWS-GFP, which contains a GFP fluorescent marker under the control of the CaMV 35S promoter (Yu & Birchler, 2016) as a pollen donor. As in vivo haploid induction by RWS functions by paternal genome elimination, haploid embryos can be identified by scoring the absence of GFP fluorescence. We first examined haploid induction using the public maize inbred line B104, which is often used for transformation and gene editing by the research community (Frame et al., 2006; Aesaert et al., 2022; Kang et al., 2022), as the maternal donor. In addition to the HI genotype, the maternal donor genotype together with environmental conditions also influences the HIR (Kalinowska et al., 2019). In 38 independent crosses between B104 (female) and RWS-GFP (male), we obtained on average 84 embryos per cross (FIG. 1a), of which 15.1% were haploid based on the absence of GFP fluorescence (FIG. 1b). Because maize haploid plants are sterile, their genomic content needs to be doubled to generate fertile diploids, i.e. doubled haploids (DH; DH0 is the first DH generation, DH1 its progeny after self-pollination). Colchicine-mediated doubling of maize haploid seedlings is routinely used in large-scale DH breeding programs, but because only 10-30% of seedlings yield DH1 seed, it is not very efficient (Chaikam et al., 2019; Molenaar et al., 2019). These routine strategies require whole or partial submersion of seedlings in a colchicine solution, which complicates the containment and safe handling of this toxic chemical. To simplify and quicken the doubling procedure, we used ERD, a method that combines embryo rescue with colchicine treatment (Barton et al., 2014; McCaw et al., 2021). Haploid immature maize embryos were placed in vitro on basic plant medium containing colchicine followed by germination. On average, 78.4% of isolated B104 embryos survived ERD and germinated in vitro (FIG. 1c). After transferring germinated plantlets to the growth chamber, flow cytometry analysis using material of the third or fourth leaf showed that, on average, 55.4% of plants were diploid and an additional 36.8% were mosaic for doubling (mixoploid) (FIG. 1d). Haploid reproductive cells cannot undergo meiosis, therefore, gametes will not be derived from haploid tissue (Chalyk, 1994). Mixoploid plants resulting from incomplete haploid doubling have a mixture of haploid and diploid tissue, but any gametes will be derived from diploid tissue (Chaikam et al., 2019). We retained all plants scored as non-haploid (DH0) for future experiments. In conclusion, we were able to set up an efficient pipeline for haploid induction in B104 by crossing with the RWS-GFP HI line, followed by ERD, yielding on average 9.6 DH0 plants per cross.

### 2.HI-BREEDIT, a Strategy to Combine Multiplex Gene Editing and Doubled Haploid Breeding

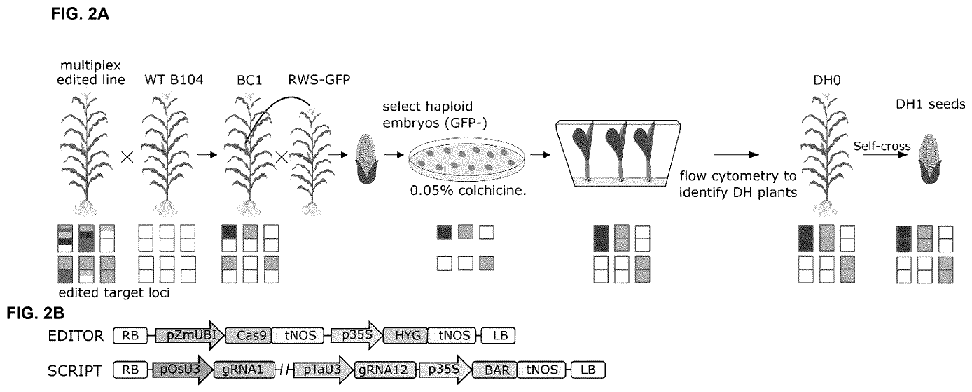

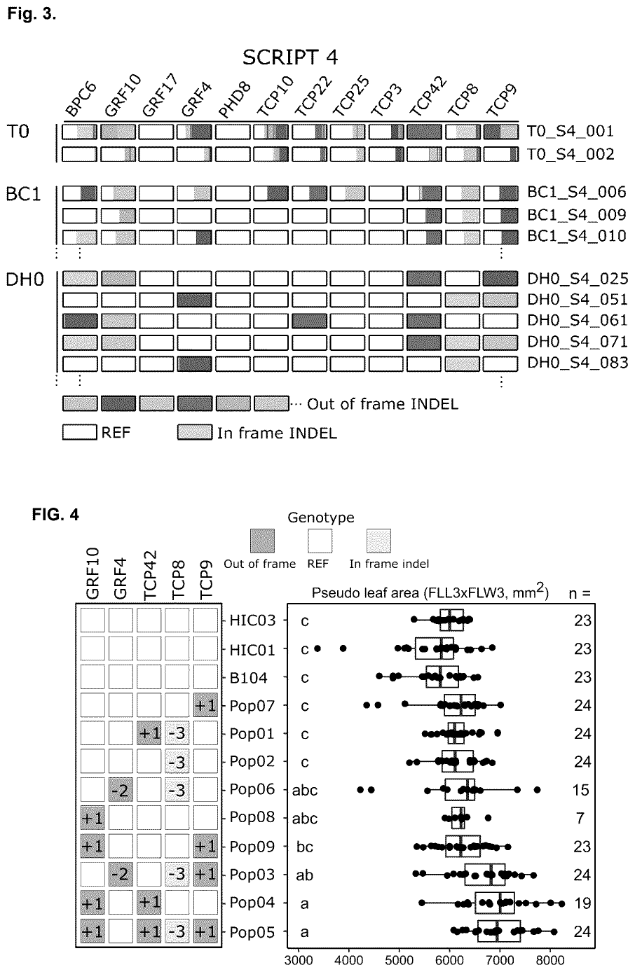

To evaluate the concept of combining DH technology and multiplex gene editing (FIG. 2a), we used multiplex CRISPR/Cas9 genome-edited B104 maize plants obtained from the BREEDIT gene discovery pipeline (Lorenzo et al., 2023). These were generated by supertransforming immature embryos that are heterozygous for a Cas9-expressing T-DNA (EDITOR 1; FIG. 2b) with different SCRIPT constructs each containing 12 gRNAs (FIG. 2b) (Lorenzo et al., 2023). We determined genotypes at target loci of primary transformants (TO plants) by highly multiplex amplicon sequencing (HiPlex) followed by read-backed haplotyping to call edited alleles (Schaumont et al., 2022). The TO plants were homozygous, heterozygous, bi-allelic, or genetic mosaic at multiple targeted loci (Lorenzo et al., 2023) (FIG. 3).

We backcrossed TO plants to wild-type B104 and the first backcross (BC1) generation plants were used as the starting material for haploid induction and ERD (FIG. 2a). Because the BC1 plants only contained heterozygous mutations, each gamete had a 50% probability of containing an edited allele at a given target locus. Hence, every gamete and resulting DH plant was expected to have a random combination of genetically unlinked edited loci. As no loci are heterozygous in DH0, the occurrence of a particular homozygous genotype of interest for n loci by self-crossing is reduced from 1/4ⁿ to 1/2ⁿ. Self-crossing DH0 plants results in DH1 seeds with identical genotypes (FIG. 2a) which are suitable for replicated phenotyping.

We selected two TO plants (T0_S4_001 and T0_S4_002) containing SCRIPT 4 with edits in ten of the twelve targeted genes (FIG. 3) (Lorenzo et al., 2023). SCRIPT 4 targets seven class II CINCINNATA-TEOSINTE BRANCHED 1/CYCLOIDEA/PROLIFERATING CELL FACTOR (TCP) genes, and also GROWTH REGULATING FACTOR4 (GRF4), GRF10 and GRF17, BASIC PENTACYSTEINE 6 (BPC6) and PHD8, a gene encoding a plant homeodomain-finger protein. These are all related to regulators of cell division, leaf shape, and leaf size determination. In the BREEDIT pipeline, populations edited with SCRIPT 4 showed strong final leaf 3 length (FLL3) and final leaf 3 width (FLW3) phenotypes. Although causative gene combinations are still unknown, the involvement of GRF10 and various TCPs was hypothesized. All twelve SCRIPT 4 gRNAs were active and also in the TO lines used here evidence of editing could be found in the sampled TO leaves for all genes but GRF17 and PHD8 (FIG. 3) (Lorenzo et al., 2023). Genotypes at each edited target locus ranged from homozygous for TCP42 and bi-allelic edits for GRF10 and TCP9 to genetic mosaic for TCP10. We backcrossed TO plants with wild-type B104 plants and 16 BC1 plants (eleven originating from T0_S4_001, five from T0_S4_002) were genotyped with HiPlex and scored for resistance to hygromycin (EDITOR T-DNA) and phosphinothricin (SCRIPT T-DNA). Genotyping confirmed the inheritance of edited alleles from TO plants because Cas9-negative and/or SCRIPT-negative plants showed heterozygous edits with identical alleles also present in TO (FIG. 3). In addition, for BC1 plants that had both the SCRIPT 4 and EDITOR T-DNA, we observed transgenerational gene editing of the inherited wild-type B104 alleles (e.g. plant BC1_S4_006; FIG. 3). We now also detected edits in GRF17 and PHD8 for which no mutations were observed in TO. We used these 16 BC1 plants and one wild-type B104 plant for haploid induction using RWS-GFP. In total, we obtained 173 plantlets after ERD, of which 165 non-haploids were sampled for genotyping (FIG. 3). As expected, the majority of DH0 plants were homozygous at all targeted loci (FIG. 3). As loci were mostly heterozygous in BC1, wild-type, and mutant alleles had an equal chance of being inherited for each genetically unlinked locus. For SCRIPT 4, only two target loci (GRF4 and PHD8) are genetically linked, and will not segregate independently (Lorenzo et al., 2023). We indeed observed a variety of edited locus combinations in DH0 plants (FIG. 3). Twenty-four out of 165 DHOs showed additional transgenerational editing and were not used for future experiments. DH0 plants were transferred to the greenhouse for self-pollination to generate DH1 seeds. In total, 105 plants successfully yielded DH1 seeds. Of these DH1 populations, 29 contained unique homozygous edited combinations involving ten out of the twelve genes, with only edits in GRF17 and TCP3 lacking. In conclusion, we confirmed that haploid induction and genome doubling can be performed using SCRIPT 4 edited plants, yielding a variety of homozygous DH populations with various combinations of edits.

3.Phenotyping SCRIPT 4 DH1s Unveils Combinations of Gene Edits with Increased Leaf Size

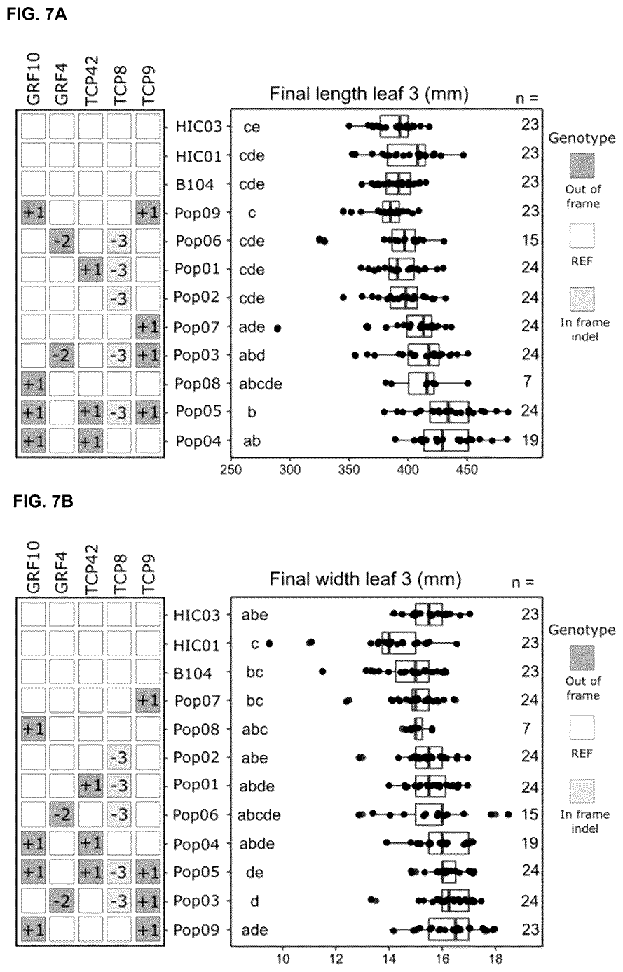

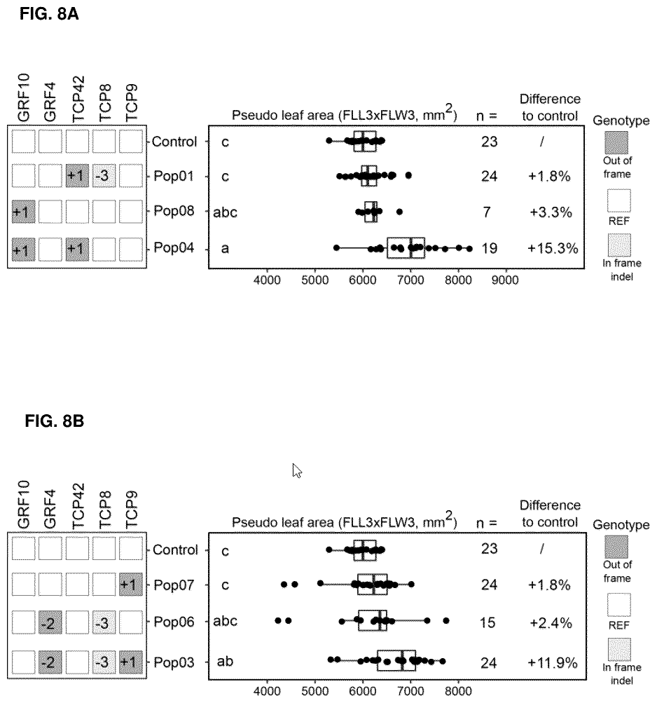

We selected nine DH1 populations derived from self-pollinated homozygous DH0 plants, with unique combinations that had a sufficient number of seeds available for phenotyping. We also included a wild-type B104 and two haploid-induced populations that only inherited wild-type alleles and went through the same procedures as the edited populations, as haploid-induced controls (HICs). We measured leaf phenotypes of DH1 plants as most target genes were selected to impact leaf growth. At V3 stage, final leaf 3 blade (FLB3), final leaf 3 length (FLL3), and final leaf 3 width (FLW3) were measured and the pseudo leaf 3 area (PLA3, FLW3xFLL3) was calculated. Measurement of PLA is fast and non-destructive and the parameter correlates with total leaf area (Pearce et al., 1975). Three edited DH1 populations displayed a significant increase in PLA3 compared with the controls (FIG. 4): Pop03 (grf4;tcp8;tcp9, on average 10.6% larger than HIC03), Pop04 (grf10;tcp42, on average 13.2% larger), and Pop05 (grf10;tcp42;tcp8;tcp9, on average 14.2% larger). Looking at FLL3, Pop04 (grf10;tcp42) and Pop05 (grf10;tcp42;tcp8;tcp9) were significantly different from the controls with an average increase of 8.2% and 8.1%, respectively compared with HIC01 (FIG. 7a). Further, for FLW3, only Pop03 (grf4;tcp8;tcp9) showed a significant increase compared with the controls (on average 5.4% larger than HIC03) (FIG. 7b). With three different gene combinations having an increased PLA3, it is clear that the main driver for this increase is FLL3 for Pop04 and Pop05, while for Pop03, FLW3 is clearly at the basis of the increased PLA3. From these results, the gene combination grf10;tcp42 seemed to be the common denominator to have a positive effect on FLL3 (FIG. 7a). Because our method generates many different gene combinations, we were also able to include single mutants of the genes involved, a valuable asset to untangle the involved genes. Here, we observed no leaf phenotypes for grf10 (Pop08) or tcp42 (with a tcp8 in-frame allele, Pop01) single out-of-frame mutants. FIG. 8 shows a detailed analysis of the pseudo leaf area of the single edited plants (GRF4 and TCP9) as compared to the combination (GRF4 x TCP9) and clearly shows the synergism of the combination (>15% pseudo leaf area increase). In addition, FIG. 8 shows a detailed analysis of the pseudo leaf area of the single edited plants (GRF10 and TCP42) as compared to the combination (GRF10 x TCP42) and clearly shows the synergism of the combination (>11% pseudo leaf area increase). In conclusion, SCRIPT 4-edited DH plants were successfully used for replicated phenotyping, which revealed three stable edited gene combinations with an increased PLA3.

### 4. Inter-SCRIPT Crosses Expand Edited Gene Combinations Even Further

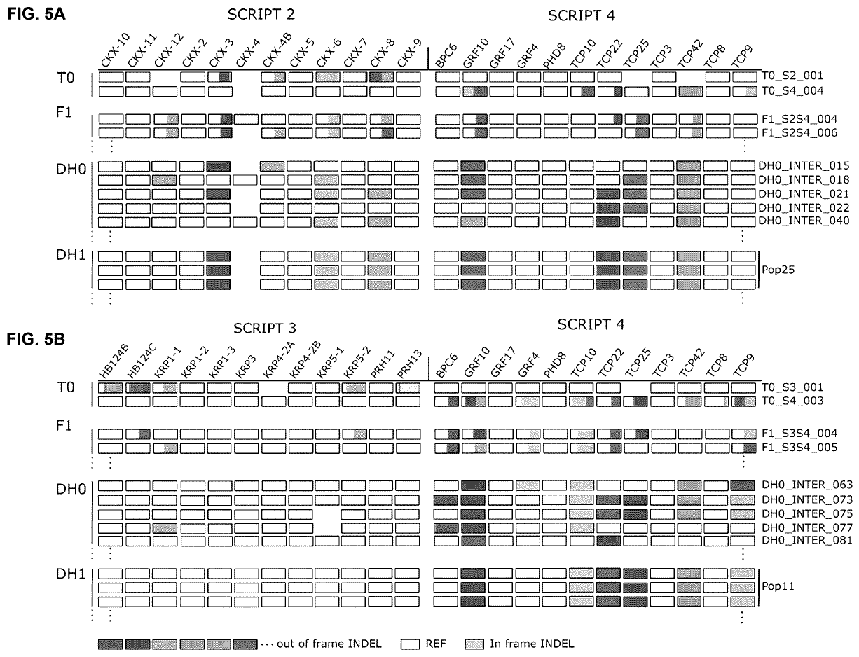

To enlarge the possible combinations of gene-edited loci even further, we combined SCRIPT 4 with two other previously generated SCRIPTs (Lorenzo et al., 2023). The different SCRIPT-targeted genes were distributed over the ten maize chromosomes with limited genetic linkage (Lorenzo et al., 2023). SCRIPT 2 targets 12 members of the cytokinin oxidase (CKX) family and SCRIPT 3 targets 12 cell cycle- and drought-related genes. As the SCRIPTs target different genes, TO plants for SCRIPT 4 are WT for SCRIPT 2- or SCRIPT 3-targeted genes and vice versa. TO plants were selected with evidence for editing in 18 of the 36 targeted genes, again ranging from homozygous edited loci (e.g. for CKX6) to mostly WT, like for TCP8 (FIG. 5). TO plants containing SCRIPT 2 were crossed with those containing SCRIPT 4 (inter-script cross, S2xS4) to yield an F1 ‘inter-script’ population with (heterozygous) edits in potentially up to 24 target loci. Similarly, SCRIPT 3 and SCRIPT 4 TO plants were crossed to yield a second F1 inter-script population (S4xS3). In this experiment, we tested F1 plants for the absence of SCRIPT and/or EDITOR T-DNA to avoid transgenerational CRISPR/Cas9 editing further down the pipeline. Subsequently, we selected six F1 plants from each inter-script F1 population that lacked the Cas9-containing EDITOR T-DNA.

HiPlex amplicon sequencing confirmed that plants contained heterozygous mutations or wild-type alleles at the targeted loci (FIG. 5). Twelve F1 plants were pollinated with RWS-GFP pollen and resulting haploid embryos were subjected to the ERD procedure. 138 haploid embryos were identified and treated with colchicine (HIR=15.3%), and 123 survived treatment (survival=89.1%). After flow cytometry analysis, 87 DH0 plants were transferred to the greenhouse for self-pollination.

Similarly, as with SCRIPT 4 alone, 73 out of 87 inter-script DH0 plants contained a variety of combinations of homozygous edits and wild-type alleles, but now spanning two SCRIPTs (FIG. 5). Fourteen out of 87 DH0 plants did not exclusively contain homozygous mutations; several heterozygous and bi-allelic mutations were observed. We attribute this to either accidental self-pollination of F1 plants or misidentification of GFP absence. Hence these DHOs were removed from further phenotypic analysis. Confirmed DH0 plants were grown to maturity in the greenhouse and plants that simultaneously formed silks and pollen at the correct interval were self-crossed. In total, we obtained DH1 progeny for 44 independent DH0 plants, including 33 unique homozygous edited combinations. To summarize, we were able to create homozygous populations from inter-script F1 plants using haploid induction and ERD. We obtained populations with up to seven homozygous out-of-frame edits suitable to be used in replicated phenotyping.

### 5.a Genetically Stable Ckx3;6;8 Tcp22;25;42 Grf10 Septuple Mutant Reproducibly Shows Increased Leaf Size

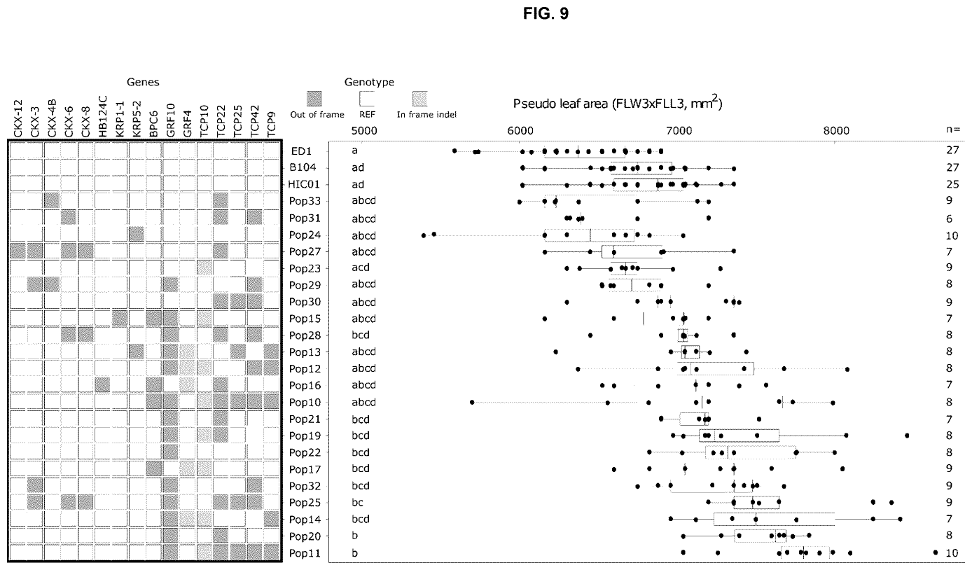

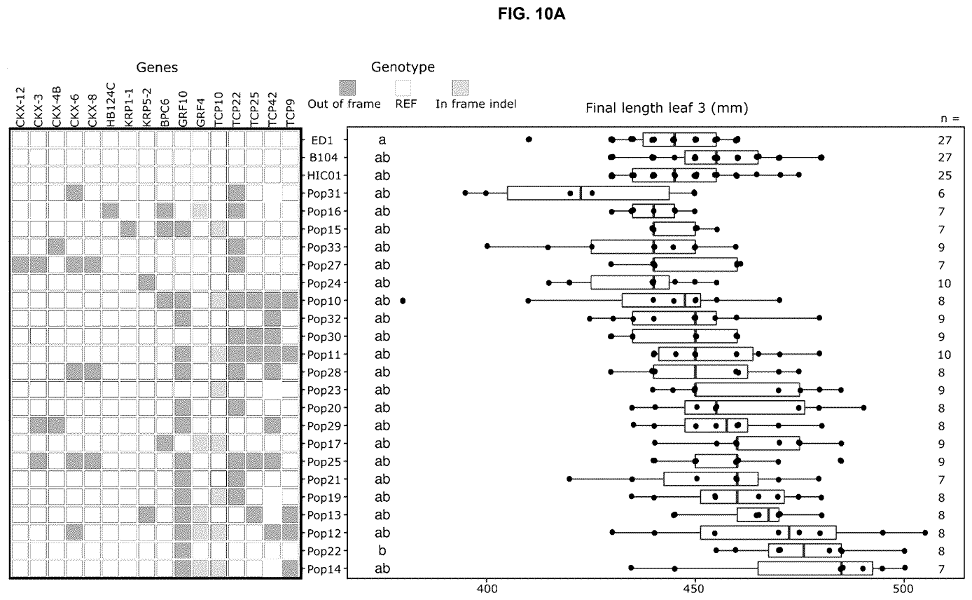

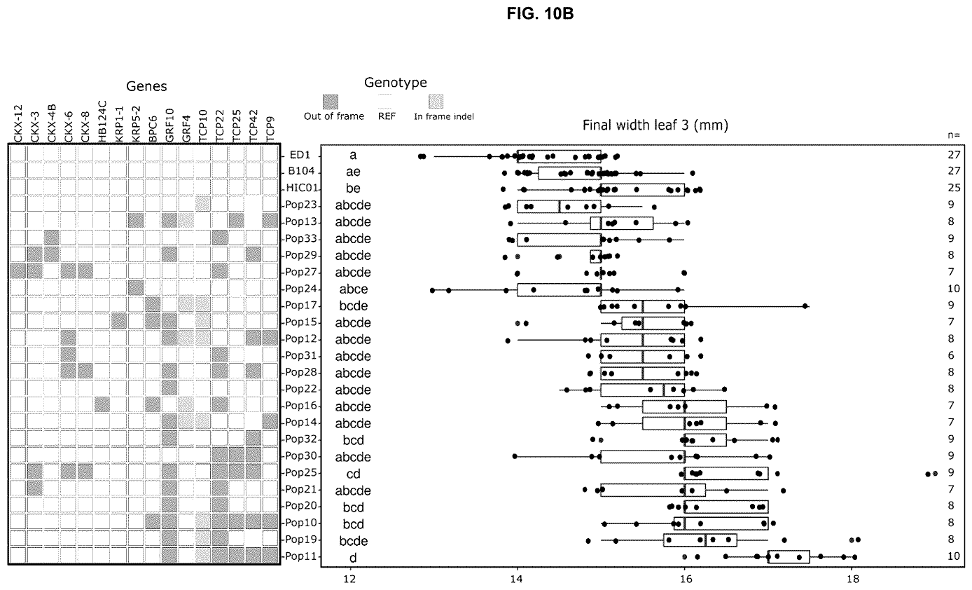

Similar to SCRIPT 4, we used a selection of inter-script DH1 populations for replicated phenotyping. To screen many populations simultaneously, 6 to 10 replicates per DH1 population were phenotyped for PLA3, demonstrating that several populations indeed showed a significantly increased PLA3 (Pop11, Pop20, and Pop25, FIG. 9). For FLL3, no populations were found to be significantly different compared with the controls (FIG. 10a), and for FLW3, two populations showed a significant increase (Pop11 and Pop25, FIG. 10b). To confirm the stability of the genotypes, we sequenced the DH1 plants of the three populations with the best PLA3 performance, and eight HIC individuals (FIG. 5). Since all sequenced DH1 individuals showed homozygous genotypes, we could conclude that DH1 populations originating from homozygous DH0 plants are genetically stable.

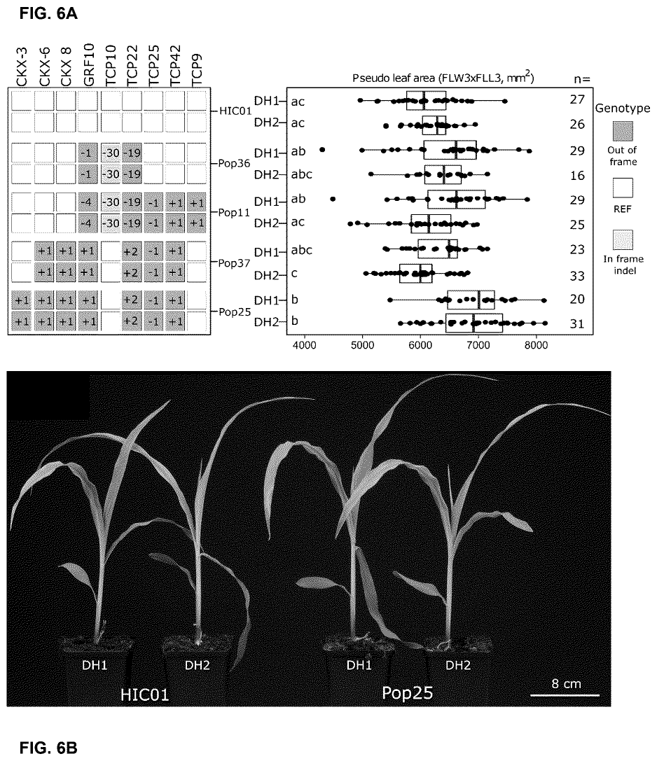

Using a power analysis for PLA3, we estimate that at least 12 genetically identical plants are needed to significantly attribute a 10% difference in PLA3 with 80% statistical power. We selected four genotypes based on the preliminary phenotypic screen for a repeated experiment, now with more individuals per population. In general, DH0 B104 plants growing in the greenhouse appeared to be smaller, often lacked proper ear and tassel development, or had a desynchronized ear and pollen maturity, leading to a lower reproduction rate and lower seed set compared with plants that did not undergo colchicine treatment. We hypothesized that this poor DH0 performance might also impact its DH1 progeny in phenotyping experiments, adding variability. Hence, for comparison, we also included corresponding DH2 plants, generated by self-crossing a DH1 plant (FIG. 6a). Four populations were selected alongside the HIC01 control, including ckx3;6;8 tcp22;25;42 grf10 (Pop25) and tcp9;10;22;25;42 grf10 (Pop11), which performed well in the preliminary phenotypic screen of the inter-script DH1s (FIGS. 9, 10b), and also two new genotypes, tcp10;22 grf10 (Pop36) and ckx6;8 tcp22;25;42 grf10 (Pop37); Pop37 only differed from Pop25 by the absence of an edit in CKX3. For each of the four selected genotypes and the HIC, a representative DH1 plant was genotyped, again confirming genetic stability. For all four lines and the HIC01, there was no significant difference in PLA3 or FLL3 between the respective DH1 and DH2 populations (FIGS. 6, 11a). For FLW3, there was however a significant difference between DH1 and DH2 for Pop11 and Pop37 (FIG. 11b). For both DH1 and DH2, the septuple mutant ckx3;6;8 tcp22;25;42 grf10 (Pop25) had a strongly increased PLA3 compared with HIC01, 13.1% higher for DH1 and 10.8% higher for DH2. Interestingly, Pop37, which only differs from Pop25 in lacking the ckx3 mutation, does not have a significantly increased PLA3, FLL3, or FLW3 compared with the control (FIG. 6, 11). This suggests a critical role for ckx3 in the expression of this phenotype.

In conclusion, DH1 generation plants can already be used for screening PLA3, and in only three generations, we established a collection of higher-order mutants, of which a septuple ckx3;6;8 tcp22;25;42 grf10 mutant reproducibly showed an enlarged PLA3.

### 6. Phenotyping of Adult Plants

For adult phenotyping experiments, DH2 seeds of grf10, tcp42, grf10 tcp42 double mutant and a wild-type doubled haploid (HIC03) were germinated in controlled growth room conditions (300 μE·m⁻²·s⁻¹, 16 h light (23° C.), and 8 h dark (22° C.) in pots containing professional potting mixture (Van Israel nv). After 12 days, plants were transferred to 10 L pots with professional potting mixture (Van Israel nv) containing controlled release fertilizer (2.0 kg·m⁻³, Osmocote®, NPK Dec. 14, 2024) and moved to the greenhouse (16 h light (25° C.), and 8 h dark (22° C.) in a full randomized design.

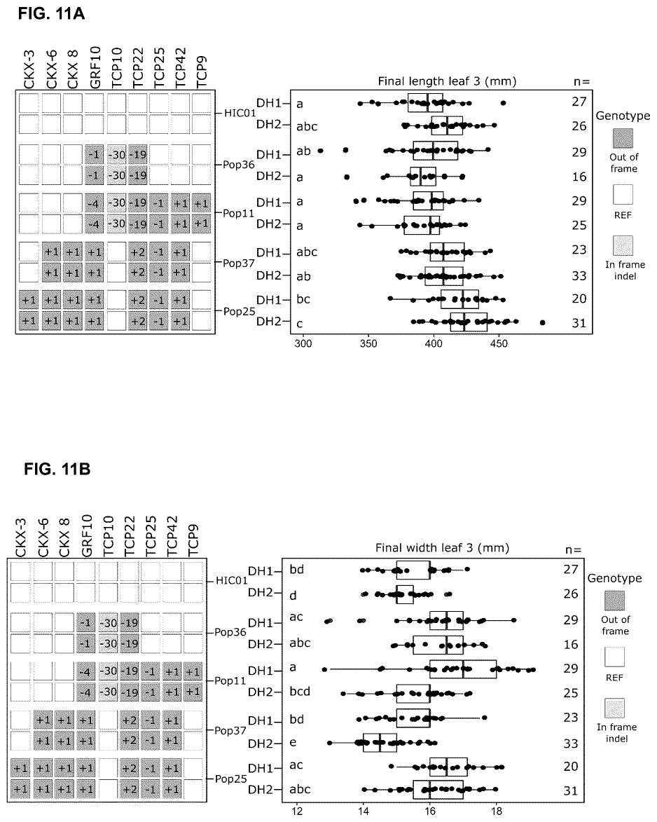

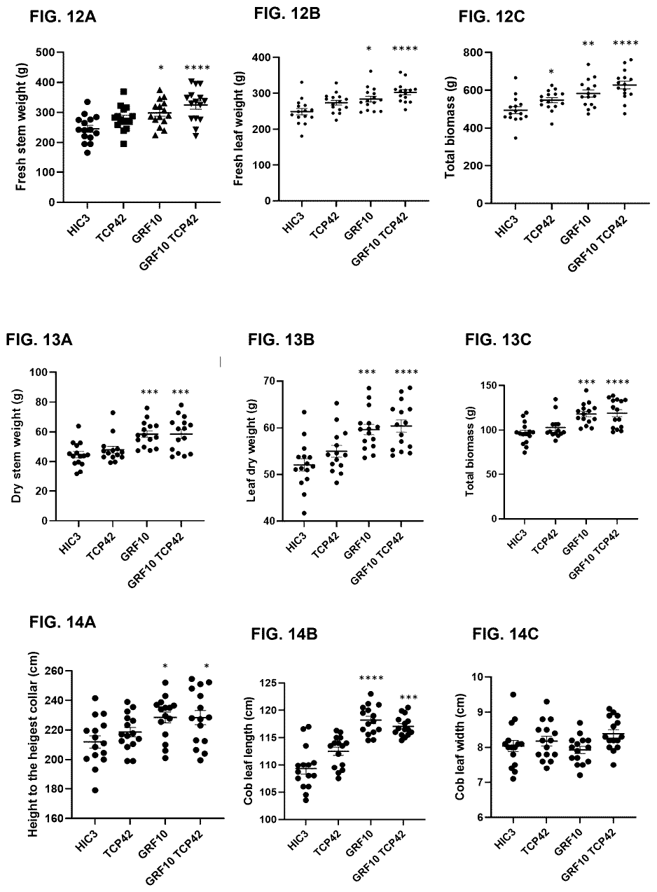

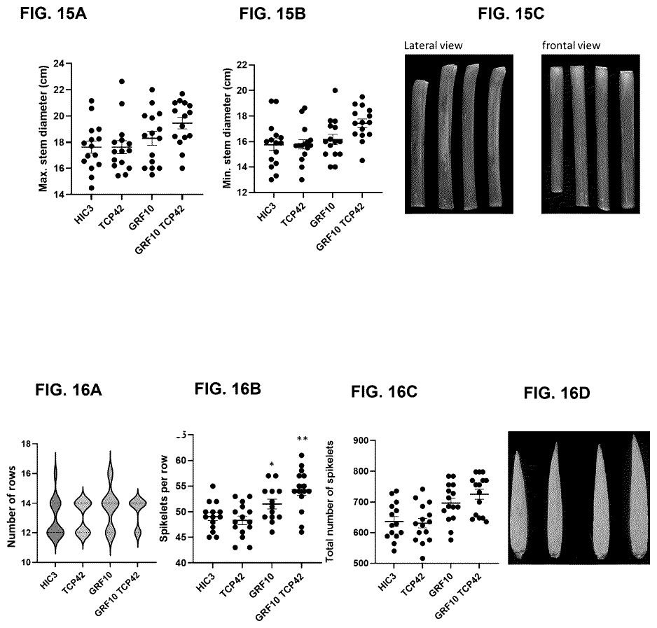

For each plant, phenotypes were recorded after silking (appearance of the first silk). First, height of the plants was determined by measuring the distance from the crown to the highest collar. Next, the lamina length (measured from the ligule to the tip) and the width (measured at the middle point) of the leaf under the primary developing ear was measured. The maximal and minimal diameter of the stem was measured at the node of the primary cob. Aboveground plant parts were harvested and split in stem, leaves and the immature ear. Fresh weight of stem and leaves was immediately determined by weighing while dry weight was determined after drying the samples in an industrial oven at 60° for one week. For measuring seed yield potential, immature ears were removed from plants after silking and the number of spikelets per row and the number of rows was determined. Pictures from immature ears were taken from ears harvested at the same silking date. Fresh biomass parameters of adult plants are depicted in FIG. 12. Dry biomass parameters of adult plants are depicted in FIG. 13. Height and leaf parameters of adult plants are depicted in FIG. 14. Stem parameters of adult plants are depicted in FIG. 15. Seed yield potential of adult plants is depicted in FIG. 16.

### SEQUENCE LISTING

### GRF10 and GRF4 Orthologous Genes

Zea mays, Zm00001eb104790, GRF4, SEQ ID NO: 7
Zea mays, Zm00007a00051377, GRF10, SEQ ID NO: 8
Arabidopsis thaliana, AT2G22840, SEQ ID NO: 9
Arabidopsis thaliana, AT4G37740, SEQ ID NO: 10
Glycine max, Glyma.14G089093, SEQ ID NO: 11
Glycine max, Glyma.17G232600, SEQ ID NO: 12
Gossypium raimondii, Gorai.004G204600, SEQ ID NO: 13
Gossypium raimondii, Gorai.007G092400, SEQ ID NO: 14
Gossypium raimondii, Gorai.008G241800, SEQ ID NO: 15
Oryza sativa, Os07g0467500, SEQ ID NO: 16
Sorghum bicolor, Sobic.002G297800, SEQ ID NO: 17
Triticum aestivum, TraesCS2A03G0552300, SEQ ID NO: 18
Triticum aestivum, TraesCS2B03G0630300, SEQ ID NO: 19
Triticum aestivum, TraesCS2D03G0561800, SEQ ID NO: 20

### TCP9 Orthologous Genes

Zea mays, Zm00007a00038186, TCP9, SEQ ID NO: 21
Arabidopsis thaliana, AT2G31070, SEQ ID NO: 22
Glycine max, Glyma.06G232300, SEQ ID NO: 23
Glycine max, Glyma.13G292500, SEQ ID NO: 24
Oryza sativa, Os01g0213800, SEQ ID NO: 25
Sorghum bicolor, Sobic.003G018700, SEQ ID NO: 26
Triticum aestivum, TraesCS3A03G0323300, SEQ ID NO: 27
Triticum aestivum, TraesCS3B03G0392900, SEQ ID NO: 28
Triticum aestivum, TraesCS3D03G0312300, SEQ ID NO: 29

### TCP42 Orthologous Genes

Zea mays, Zm00001eb328570, SEQ ID NO: 30
Arabidopsis thaliana, AT3G02150, SEQ ID NO: 31
Arabidopsis thaliana, AT5G60970, SEQ ID NO: 32
Glycine max, Glyma.05G142000, SEQ ID NO: 33
Glycine max, Glyma.08G097900, SEQ ID NO: 34
Glycine max, Glyma.13G219900, SEQ ID NO: 35
Glycine max, Glyma.15G092500, SEQ ID NO: 36
Gossypium raimondii, Gorai.001G076700, SEQ ID NO: 37
Gossypium raimondii, Gorai.006G009800, SEQ ID NO: 38
Gossypium raimondii, Gorai.012G048500, SEQ ID NO: 39
Oryza sativa, Os03g0785800, SEQ ID NO: 40
Oryza sativa, Os07g0152000, SEQ ID NO: 41
Sorghum bicolor, Sobic.008G172200, SEQ ID NO: 42
Triticum aestivum, TraesCS5A03G0079200, SEQ ID NO: 43
Triticum aestivum, TraesCS5B03G0080000, SEQ ID NO: 44
Triticum aestivum, TraesCS5D03G0103300, SEQ ID NO: 45

### Materials and Methods

### Plant Material, Handling, and Growth Conditions

Seeds of the maize inbred line B104 were originally obtained from the USDA National Plant Germplasm System (Accession no. PI 594047). Single maize B104 seeds were placed in a pre-wetted Jiffy-70 pellet and kept in controlled growth room conditions (300 μE·m⁻²·s⁻¹, 16 h light (23° C.), and 8 h dark (22° C.)). The seedlings were transferred to medium-sized pots with professional potting mixture (Van Israel nv). After three weeks, plants were transferred to 10-L pots with professional potting mixture (Van Israel nv) containing controlled release fertilizer (2.0 kg/m³, Osmocote®, NPK Dec. 14, 2024) and moved to the greenhouse (16 h light (25° C.), and 8 h dark (22° C.)) until grown to maturity. Seeds of the RWS-GFP line were originally obtained from the lab of Dr. James Birchler (Yu & Birchler, 2016). Because the time from sowing until flowering is 70 days for B104 and 60 days for RWS, the moments for seed sowing were adjusted accordingly to ensure that parent (edited) B104 ears could be pollinated by RWS-GFP pollen, in our greenhouse. Emerging ears of maize plants were covered with a paper shoot bag to avoid unwanted cross-pollination. Silks of B104 female flowers were cut back (3-5 cm from the top, without nicking the ear itself) one day before pollination. On the day of pollination, a paper tassel bag was used to collect RWS-GFP pollen. Anthers and pollen were separated by shaking the bag gently and pollen was sprinkled onto the re-emerged silks of the parent B104 plant.

### Embryo Isolation and Colchicine Treatment

Fourteen days after pollination, cobs were harvested from the plants, husk leaves were removed and the cobs were surface-sterilized in 5% NaOCl+0.01% Tween® 20 (Sigma-Aldrich) solution for 2 min. Sterilized cobs were washed three times with sterile purified water. A sterile scalpel was used to cut off the top of the kernels and a small sterile spatula was used to isolate all embryos from the cob. Embryos were all collected on a square Petri dish with basic plant medium (Regeneration II medium (Aesaert et al., 2022)). This medium was also used for colchicine treatment and subsequent germination of the embryos. 1 L medium is composed of 4.3 g MS salts, 30 g sucrose, 100 mg myo-inositol, pH 5.8, then 3 g gelrite was added and the medium was autoclaved. After autoclaving, 1 ml of a 1000× stock of MS vitamins was added. Colchicine treatment of embryos was based on the method described by Barton et al. (2014) with modifications. Fluorescent microscopy was used to separate GFP-expressing diploid embryos from non-GFP-expressing haploid embryos. Diploid embryos were discarded and haploid embryos were moved onto plates with the plant medium described above, supplemented with 0.05% colchicine and 0.5% DMSO (colchicine was dissolved in DMSO and the mixture was added to the medium after autoclaving). Haploid embryos were put with the scutellum facing upwards onto the colchicine-containing medium spaced at least a few mm apart and incubated for 24 h in the dark at 25° C. Treated embryos were moved to Sterivent high containers (107×94×96 mm, Duchefa, Haarlem, The Netherlands) containing Regeneration II medium without colchicine and placed with the scutellum facing down. Embryos were incubated for approximately six days in the dark at 25° C.; etiolated roots and shoots emerge from the embryos at this stage. After six days, the Sterivent containers were moved to light conditions (80-100 μE·m⁻²·s⁻¹, 16 h light (24° C.), and 8 h dark (22° C.)) where they grew into green plantlets. The plantlets were then transferred to a pre-wetted Jiffy-7 pellet and covered with a plastic box to maintain high humidity; facilitating the transition from tissue culture to the growth chamber. Plantlets were kept in controlled growth room conditions (300 μE·m⁻²·s⁻¹, 16 h light (25° C.), and 8 h dark (22° C.)). The humidifying cover was removed after three days. After 2-3 weeks, plantlets were tested by flow cytometry. Selected plants were transferred to larger pots and moved to the greenhouse until maturity, as described above.

### Flow Cytometry Analysis

Flow cytometry analysis was used to assess the ploidy levels of the colchicine-treated plants. Approximately 1 cm² of leaf tip material (leaf 3-4) was cut from the plants and chopped into fine pieces using a razor blade in 200 μL chilled CyStain UV Precise P Nuclei Extraction Buffer (Partec) and supplemented with 800 μL chilled CyStain UV Precise P Nuclei Staining Buffer (Partec). The mixture was filtered through a 50-μm filter and analyzed with a CyFlow®ML cytometer (Partec). An untreated, diploid plant was used as a control. The DNA content distribution of the nuclei was analyzed using FloMax (Windows™) and/or the Floreada web tool (https://floreada.io/analysis).

### Genomic DNA Isolation and Multiplex Amplicon Sequencing

A piece of 1-2 cm of leaf material was placed in 8-strip, 2-mL capacity tubes (National Scientific Supply Co) together with two 3-mm stainless steel ball bearings, snap frozen in liquid nitrogen, and ground using a Mixer Mill MM400 (Retsch®). 0.5 mL DNA extraction buffer (2.5 mL 1 M Tris-HCl pH 8, 3 mL 5 M NaCl, 5 g saccharose, to 50 mL with Milli-Q water) was added, and samples were shaken and incubated at 65° C. for 20 min. Tubes were centrifuged (2 min at 1800×g) and 50 μL of supernatant was mixed with 70 μL magnetic beads (HighPrep™ PCR Clean-up System, Magbio) and put on a magnet. The supernatant was taken off, beads were washed twice with 80% ethanol and dried for further processing. Highly multiplex amplicon sequencing (HiPlex, Floodlight Genomics LLC, Knoxville, TN, USA) was performed as described in Lorenzo et al. (2023).

### Phenotyping

Phenotyping analysis was performed as described in Lorenzo et al. (2023). For the phenotypic experiments, only well-watered conditions were used (2.4 g of water per gram of dry potting mix, replenished three times a week). Plants were grown in the growth chamber (23° C.) under a long-day photoperiod (16 h day, 8 h night). Phenotypic traits were measured for all plants at V3 (when the collar of leaf 3 was fully developed). Final leaf 3 length (FLL3) was measured from the crown of the plant to the leaf tip, final leaf 3 blade length (FLB3) was measured from the collar to the leaf tip, and final leaf 3 width (FLW3) was measured at the widest point of the leaf blade. Sheath length (SL3) and pseudo leaf area (PLA3) are calculated based on other measurements: SL3=FLL3-FLB3, PLA3=FLL3xFLW3. All statistical tests were performed at a significance level of 5%. Normality was assessed using the Shapiro-Wilk Normality test and Q-Q plots. The equality of variances was studied with Levene's test. The Kruskal-Wallis rank sum test was used to find differences within groups followed by a Wilcoxon rank sum test (p-values were adjusted using the Holm correction (Holm, 1979)). Data analysis was performed using R (version 4.0.3) with the packages; stats and car.

TABLE 1
sequences of the sgRNA spacers used to generate loss-of function
alleles in the Zea mays genes

| GENE | SCRIPT | NAME | sgRNA spacer |
|---|---|---|---|
| Zm00007a00030149 | S1 | D8 | TCGAGGAGGGAGCTGTCCGGTGG (SEQ ID NO: 46) |
| Zm00007a00018017 | S1 | D9 | GGAGGGAGCCGTCCGGTGCCGGG (SEQ ID NO: 47) |
| Zm00007a00029741 | S1 | Ga2ox13 | AGGCAGGGGTAGTTCAGCGCCGG (SEQ ID NO: 48) |
| Zm00007a00019492 | S1 | Ga2ox2 | TATATGCAGGGACGTGGTGCAGG (SEQ ID NO: 49) |
| Zm00007a00026788 | S1 | Ga2ox4 | GGCGCGATGTCAAAGCTGGCCGG (SEQ ID NO: 50) |
| Zm00007a00020286 | S1 | Ga2ox5 | CGGACGGGTCAGCCGGCACCTGG (SEQ ID NO: 51) |
| Zm00007a00048908 | S1 | Ga2ox7 | TGACGGGACAGGGGAGGCCTTGG (SEQ ID NO: 52) |
| Zm00007a00003769 | S1 | Ga2ox8 | CGACGAGATCTTCACGGTGTTGG (SEQ ID NO: 53) |
| Zm00007a00048936 | S1 | Ga2ox9 | CCGCGCCGGCCGGGCCGTTACGG (SEQ ID NO: 54) |
| Zm00007a00004590 | S1 | SLRL1-1 | ACGGCGGCCCAATGCCCGTGAGG (SEQ ID NO: 55) |
| Zm00007a00034449 | S1 | SLRL2 | GTACACCTCTGTGAGTGAGTCGG (SEQ ID NO: 56) |
| Zm00007a00007620 | S1 | SPY | GATAACTCCCATGTTGCAATAGG (SEQ ID NO: 57) |
| Zm00007a00010979 | S2 | CKX-10 | AGGCCCCGCACGTGCTTGAGCGG (SEQ ID NO: 58) |
| Zm00007a00046273 | S2 | CKX-11 | AGCTCAGCTGCCCCGAGACCAGG (SEQ ID NO: 59) |
| Zm00007a00035050 | S2 | CKX-12 | TTGGGCGGAGCAACTTGCAGCGG (SEQ ID NO: 60) |
| Zm00007a00000457 | S2 | CKX-2 | ATAGCACTCTTCCATCTGACTGG (SEQ ID NO: 61) |
| Zm00007a00009518 | S2 | CKX-3 | TGAGACGCTCAAGCACGGTCTGG (SEQ ID NO: 62) |
| Zm00007a00031769 | S2 | CKX-4 | CTTGAGCGAGGTGAGCTTCACGG (SEQ ID NO: 63) |
| Zm00007a00037930 | S2 | CKX-4B | TGTTGTGGCTGTTACCCTTTGGG (SEQ ID NO: 64) |
| Zm00007a00036798 | S2 | CKX-5 | TTCCTGGACCGCGTGAGCGCCGG (SEQ ID NO: 65) |
| Zm00007a00045990 | S2 | CKX-6 | CTTCCTGAACCGGGTCAGGATGG (SEQ ID NO: 66) |
| Zm00007a00024305 | S2 | CKX-7 | TGTTCATGGCAGCTCTAGGCGGG (SEQ ID NO: 67) |
| Zm00007a00001643 | S2 | CKX-8 | GAACTCGGACCTGTTCATGGCGG (SEQ ID NO: 68) |
| Zm00007a00051137 | S2 | CKX-9 | CACCCAATGCTGCGTAGAACAGG (SEQ ID NO: 69) |
| Zm00007a00052555 | S3 | HB124B | CATGGAGGATATCCACGCACCGG (SEQ ID NO: 70) |
| Zm00007a00030294 | S3 | HB124C | AGGTCATTGACTCAGTCCACTGG (SEQ ID NO: 71) |
| Zm00007a00050006 | S3 | KRP1-1 | CTCGCGGTCTGGGCCGCTCCCGG (SEQ ID NO: 72) |
| Zm00007a00002369 | S3 | KRP1-2 | TGTCGTCACCGCGAGCAACTCGG (SEQ ID NO: 73) |
| Zm00007a00028283 | S3 | KRP1-3 | CGCAGAAGCTGTGCTCGCGACGG (SEQ ID NO: 74) |
| Zm00007a00022719 | S3 | KRP3 | CTTACGCCTCTGCGAATCGCCGG (SEQ ID NO: 75) |
| Zm00007a00034010 | S3 | KRP4-2A | CTCCTCACGGACGCCACCTGCGG (SEQ ID NO: 76) |
| Zm00007a00024083 | S3 | KRP4-2B | CCCGACCTCCTCGTGGTCGCCGG (SEQ ID NO: 77) |
| Zm00007a00045940 | S3 | KRP5-1 | CCGCACGCTCGCGCTGCAGAGGG (SEQ ID NO: 78) |
| Zm00007a00023470 | S3 | KRP5-2 | CGGTATGAACCGGCAGCTCGGGG (SEQ ID NO: 79) |
| Zm00007a00018143 | S3 | PRH11 | GGCTCGCCCATGCACTTCTTCGG (SEQ ID NO: 80) |
| Zm00007a00045770 | S3 | PRH13 | GACACTCGTGCCAGAACCTGTGG (SEQ ID NO: 81) |
| Zm00007a00000970 | S4 | BPC6 | TTCTTTGTTCCATTGAGAGGAGG (SEQ ID NO: 82) |
| Zm00007a00051377 | S4 | GRF10 | GGACCTCCGGATCCTCGACCAGG (SEQ ID NO: 83) |
| Zm00007a00016162 | S4 | GRF17 | TTCTGTCCTGTTCGCAGGAGAGG (SEQ ID NO: 84) |
| Zm00007a00004126 | S4 | GRF4 | CATCCGTCCCTGACGGTGCTGGG (SEQ ID NO: 85) |
| Zm00007a00007962 | S4 | PHD8 | CACTGGACATATGAACAAACCGG (SEQ ID NO: 86) |
| Zm00007a00037498 | S4 | ZmTCP10 | TGCCCAACCATGAGGTCCTGCGG (SEQ ID NO: 87) |
| Zm00007a00038701 | S4 | ZmTCP22 | CCAGTTCCCGCCGCAAGGGCAGG (SEQ ID NO: 88) |
| Zm00007a00010983 | S4 | ZmTCP25 | GTACCGGTCCAAGACTCGCCGGG (SEQ ID NO: 89) |
| Zm00007a00011364 | S4 | ZmTCP3 | AGGCGCGCACGGGCTGTCGACGG (SEQ ID NO: 90) |
| Zm00007a00035633 | S4 | ZmTCP42 | CTCGAAGGATATGGCCGACGCGG (SEQ ID NO: 91) |
| Zm00007a00039284 | S4 | ZmTCP8 | CAGCACTTCGGCTTGGCCAGTGG (SEQ ID NO: 92) |
| Zm00007a00038186 | S4 | ZmTCP9 | CAGCTCCTGGCTGTGTCCGCCGG (SEQ ID NO: 93) |

### REFERENCES

Aesaert S, Impens L, Coussens G, Van Lerberge E, Vanderhaeghen R, Desmet L, Vanhevel Y, Bossuyt S, Wambua A N, Van Lijsebettens M, et al. 2022. Optimized transformation and gene editing of the B104 public maize inbred by improved tissue culture and use of morphogenic regulators. Frontiers in Plant Science 13:883847.
Barton J E, Maddock S E, Wu X E, Zhao Z-Y, Williams M E, Hussain T, Gordon-Kamm W J. 2014. Doubling of chromosomes in haploid embryos (https://patents.google.com/patent/U.S. Pat. No. 8,859,846B2/en).
Bregitzer P, Dahleen L S, Neate S, Schwarz P, Manoharan M. 2008. A single backcross effectively eliminates agronomic and quality alterations caused by somaclonal variation in transgenic barley. Crop Science 48:471-479.
Budhagatapalli N, Halbach T, Hiekel S, Büchner H, Müller A E, Kumlehn J. 2020. Site-directed mutagenesis in bread and durum wheat via pollination by cas9/guide RNA-transgenic maize used as haploidy inducer. Plant Biotechnology Journal 18:2376-2378.
Chaikam V, Molenaar W, Melchinger A E, Boddupalli P M. 2019. Doubled haploid technology for line development in maize: technical advances and prospects. Theoretical and Applied Genetics 132:3227-3243.
Chalyk S T. 1994. Properties of maternal haploid maize plants and potential application to maize breeding. Euphytica 79:13-18.
Chang M-T, Coe E H. 2009. Doubled haploids. Biotechnology in Agriculture and Forestry 63:127-142.
Coe Jr E H. 1959. A line of maize with high haploid frequency. American Naturalist 93:381-382.
Corcoran E T, LeBlanc C, Huang Y-C, Arias Tsang M, Sarkiss A, Hu Y, Pedmale U V, Jacob Y. 2022. Systematic histone H4 replacement in Arabidopsis thaliana reveals a role for H4R17 in regulating flowering time. Plant Cell 34:3611-3631.
Cusack S A, Wang P, Lotreck S G, Moore B M, Meng F, Conner J K, Krysan P J, Lehti-Shiu M D, Shiu S-H. 2021. Predictive models of genetic redundancy in Arabidopsis thaliana. Molecular Biology and Evolution 38:3397-3414.
Devaux P. 2021. Production of wheat doubled haploids through intergeneric hybridization with maize. Methods in Molecular Biology 2287:267-279.
Dong L, Li L, Liu C, Liu C, Geng S, Li X, Huang C, Mao L, Chen S, Xie C. 2018. Genome editing and double-fluorescence proteins enable robust maternal haploid induction and identification in maize. Molecular Plant 11:1214-1217.
Frame B R, McMurray J M, Fonger T M, Main M L, Taylor K W, Torney F J, Paz M M, Wang K. 2006. Improved Agrobacterium-mediated transformation of three maize inbred lines using M S salts. Plant Cell Reports 25:1024-1034.
Gilles L M, Khaled A, Laffaire J B, Chaignon S, Gendrot G, Laplaige J, Bergès H, Beydon G, Bayle V, Barret P, et al. 2017. Loss of pollen-specific phospholipase NOT LIKE DAD triggers gynogenesis in maize. EMBO Journal 36:707-717.
Hall A E, Bleecker A B. 2003. Analysis of combinatorial loss-of-function mutants in the Arabidopsis ethylene receptors reveals that the ers1 etr1 double mutant has severe developmental defects that are EIN2 dependent. Plant Cell 15:2032-2041.
Holm S. 1979. A simple sequentially rejective multiple test procedure. Scandinavian Journal of Statistics 6:65-70.
Ingvarsson P K, Street N R. 2011. Association genetics of complex traits in plants. New Phytologist 189:909-922.
Kalinowska K, Chamas S, Unkel K, Demidov D, Lermontova I, Dresselhaus T, Kumlehn J, Dunemann F, Houben A. 2019. State-of-the-art and novel developments of in vivo haploid technologies. Theoretical and Applied Genetics 132:593-605.
Kang M, Lee K, Finley T, Chappell H, Veena V, Wang K. 2022. An improved Agrobacterium-mediated transformation and genome-editing method for maize inbred B104 using a ternary vector system and immature embryos. Frontiers in Plant Science 13:860971.
Kelliher T, Starr D, Richbourg L, Chintamanani S, Delzer B, Nuccio M L, Green J, Chen Z, McCuiston J, Wang W, et al. 2017. MATRILINEAL, a sperm-specific phospholipase, triggers maize haploid induction. Nature 542:105-109.
Kelliher T, Starr D, Su X, Tang G, Chen Z, Carter J, Wittich P E, Dong S, Green J, Burch E, et al. 2019. One-step genome editing of elite crop germplasm during haploid induction. Nature Biotechnology 37:287-292.
Liu C, Li X, Meng D, Zhong Y, Chen C, Dong X, Xu X, Chen B, Li W, Li L, et al. 2017. A 4-bp insertion at ZmPLA1 encoding a putative phospholipase A generates haploid induction in maize. Molecular Plant 10:520-522.
Liu C, Zhong Y, Qi X, Chen M, Liu Z, Chen C, Tian X, Li J, Jiao Y, Wang D, et al. 2020. Extension of the in vivo haploid induction system from diploid maize to hexaploid wheat. Plant Biotechnology Journal 18:316-318.
Liu H-J, Jian L, Xu J, Zhang Q, Zhang M, Jin M, Peng Y, Yan J, Han B, Liu J, et al. 2020. High-throughput CRISPR/Cas9 mutagenesis streamlines trait gene identification in maize. Plant Cell 32:1397-1413.
Lorenzo C D, Debray K, Herwegh D, Develtere W, Impens L, Schaumont D, Vandeputte W, Aesaert S, Coussens G, De Boe Y, et al. 2023. BREEDIT: a multiplex genome editing strategy to improve complex quantitative traits in maize. Plant Cell 35:218-238.
Mayer M, Holker A C, Presterl T, Ouzunova M, Melchinger A E, Schon C-C. 2022. Genetic diversity of European maize landraces: Dataset on the molecular and phenotypic variation of derived doubled-haploid populations. Data in Brief 42:108164.
McCaw M E, Lee K, Kang M, Zobrist J D, Azanu M K, Birchler J A, Wang K. 2021. Development of a transformable fast-flowering mini-maize as a tool for maize gene editing. Frontiers in genome editing 2:622227.
Molenaar W S, Schipprack W, Brauner P C, Melchinger A E. 2019. Haploid male fertility and spontaneous chromosome doubling evaluated in a diallel and recurrent selection experiment in maize. Theoretical and Applied Genetics 132:2273-2284.
Pearce R B, Mock J J, Bailey T B. 1975. Rapid method for estimating leaf area per plant in maize. Crop Science 15:691-694.
Piskorz E W, Xu L, Ma Y, Jiang H. 2023. Doubled-haploid induction generates extensive differential DNA methylation in Arabidopsis. Journal of Experimental Botany 74:835-847.
Röber F K, Gordillo G A, Geiger H H. 2005. In vivo haploid induction in maize. Performance of new inducers and significance of doubled haploid lines in hybrid breeding [Zea mays L.]. Maydica 50:275-283.
Schaumont D, Veeckman E, Van der Jeugt F, Haegeman A, Glabeke Sv, Bawin Y, Lukasiewicz J, Blugeon S, Barre P, de la O. Leyva-Pérez M, et al. 2022. Stack Mapping Anchor Points (SMAP): a versatile suite of tools for read-backed haplotyping. bioRxiv 2022.03.10.483555.
Schnable P S, Ware D, Fulton R S, Stein J C, Wei F, Pasternak S, Liang C, Zhang J, Fulton L, Graves T A, et al. 2009. The B73 maize genome: complexity, diversity, and dynamics. Science 326:1112-1115.
Sun G, Geng S, Zhang H, Jia M, Wang Z, Deng Z, Tao S, Liao R, Wang F, Kong X, et al. 2022. Matrilineal empowers wheat pollen with haploid induction potency by triggering postmitosis reactive oxygen species activity. New Phytologist 233:2405-2414.
Sun X, Cahill J, Van Hautegem T, Feys K, Whipple C, Novák O, Delbare S, Versteele C, Demuynck K, De Block J, et al. 2017. Altered expression of maize PLASTOCHRON1 enhances biomass and seed yield by extending cell division duration. Nature Communications 8:14752.
Vanhaeren H, Gonzalez N, Coppens F, De Milde L, Van Daele T, Vermeersch M, Eloy N B, Storme V, Inzé D. 2014. Combining growth-promoting genes leads to positive epistasis in Arabidopsis thaliana. eLife 3: e02252.
Vanhaeren H, Inze D, Gonzalez N. 2016. Plant growth beyond limits. Trends in Plant Science 21:102-109.
Verslues P E, Bailey-Serres J, Brodersen C, Buckley T N, Conti L, Christmann A, Dinneny J R, Grill E, Hayes S, Heckman R W, et al. 2023. Burning questions for a warming and changing world: 15 unknowns in plant abiotic stress. Plant Cell 35:67-108.
Wang B, Zhu L, Zhao B, Zhao Y, Xie Y, Zheng Z, Li Y, Sun J, Wang H. 2019. Development of a haploid-inducer mediated genome editing system for accelerating maize breeding. Molecular Plant 12:597-602.
Wang W, Pan Q, He F, Akhunova A, Chao S, Trick H, Akhunov E. 2018. Transgenerational CRISPR-Cas9 activity facilitates multiplex gene editing in allopolyploid wheat. CRISPR Journal 1:65-74.
Wang Y, Cheng X, Shan Q, Zhang Y, Liu J, Gao C, Qiu J-L. 2014. Simultaneous editing of three homoeoalleles in hexaploid bread wheat confers heritable resistance to powdery mildew. Nature Biotechnology 32:947-951.
Xing H-L, Dong L, Wang Z-P, Zhang H-Y, Han C-Y, Liu B, Wang X-C, Chen Q-J. 2014. A CRISPR/Cas9 toolkit for multiplex genome editing in plants. BMC Plant Biology 14:327.
Xu X, Li L, Dong X, Jin W, Melchinger A E, Chen S. 2013. Gametophytic and zygotic selection leads to segregation distortion through in vivo induction of a maternal haploid in maize. Journal of Experimental Botany 64:1083-1096.
Yao L, Zhang Y, Liu C, Liu Y, Wang Y, Liang D, Liu J, Sahoo G, Kelliher T. 2018. OsMATL mutation induces haploid seed formation in indica rice. Nature Plants 4:530-533.
Yu J, Holland J B, McMullen M D, Buckler E S. 2008. Genetic design and statistical power of nested association mapping in maize. Genetics 178:539-551.
Yu W C, Birchler J A. 2016. A green fluorescent protein-engineered haploid inducer line facilitates haploid mutant screens and doubled haploid breeding in maize. Molecular Breeding 36:5.
Zhong Y, Liu C, Qi X, Jiao Y Y, Wang D, Wang Y, Liu Z K, Chen C, Chen B, Tian X, et al. 2019. Mutation of ZmDMP enhances haploid induction in maize. Nature Plants 5:575-580.
Zhu H, Li C, Gao C. 2020. Applications of CRISPR-Cas in agriculture and plant biotechnology. Nature Reviews Molecular Cell Biology 21:661-677.

1. A cultivated crop plant or part thereof selected from:
i) a crop plant or part there of comprising a loss-of-function allele of a first endogenous gene encoding for a polypeptide comprising SEQ ID NO: 1 and 2 and comprising a loss-of-function allele of a second endogenous gene encoding for a polypeptide comprising SEQ ID NO: 3 and 4 or
ii) a crop plant or part there of comprising a loss-of-function allele of a first endogenous gene encoding for a polypeptide comprising SEQ ID NO: 1 and 2 and comprising a loss-of-function allele of a second endogenous gene encoding for a polypeptide comprising SEQ ID NO: 5 and 6.

2. the cultivated crop plant or part thereof according to claim 1 wherein the plant is homozygous for the first and second loss-of-function alleles of the endogenous genes.

3. the cultivated crop plant or part thereof of claim 1, wherein the loss-of-function allele comprises a frameshift mutation, a missense mutation, or a nonsense mutation in the endogenous genes.

4. the cultivated crop plant or part thereof of claim 1, wherein the plant has an increased yield as compared to an otherwise identical cultivated crop plant lacking a loss-of function allele in either the first or second endogenous genes.

5. the cultivated crop plant or part thereof of claim 1, wherein the cultivated crop plant is not exclusively produced by an essentially biological method.

6. the cultivated crop plant or part thereof of claim 1, wherein the part is a seed, stem or pollen.

7. the cultivated crop plant or part thereof of claim 1, wherein the cultivated crop plant or part thereof is selected from the group consisting of rice, wheat, barley, corn, soybean, cotton, sugarcane, sorghum, millet, rye, oats, cocoa, beans, grape, tomato, cassava, castor bean, poplar, eucalyptus, papaya, and oilseed.
